SEQUENCE LISTING (119 sequences)

SEQ ID NO: 1 - DNA, 45 bp, Artificial
  primer sequence of BE-Nterm-FP
ccacaacacttttgtcttatacttggccgccaccatgaaacggac

SEQ ID NO: 2 - DNA, 45 bp, Artificial
  primer sequence of BE-Nterm-RP
agttccagggggtgatggtttcctcgctctttctggtcatccagg

SEQ ID NO: 3 - DNA, 45 bp, Artificial
  primer sequence of BE-Cterm-FP
atgaccagaaagagcgaggaaaccatcaccccctggaacttcgag

SEQ ID NO: 4 - DNA, 53 bp, Artificial
  primer sequence of BE-Cterm-RP
attccatatgacgcgtcccgggatcttagactttcctcttcttcttgggctcg

SEQ ID NO: 5 - DNA, 26 bp, Artificial
  primer sequence of TRE3G-PGK-FP
gatcccgggacgcgtcatatggaatt

SEQ ID NO: 6 - DNA, 46 bp, Artificial
  primer sequence of TRE3G-PGK-RP
cgcggtgagttcaggctttttcatggtaagcttgggctgcaggtcg

SEQ ID NO: 7 - DNA, 20 bp, Artificial
  primer sequence of lenti-hygro-FP
atgaaaaagcctgaactcac

SEQ ID NO: 8 - DNA, 26 bp, Artificial
  primer sequence of lenti-hygro-RP
tcattattcctttgccctcggacgag

SEQ ID NO: 9 - DNA, 44 bp, Artificial
  primer sequence of WPRE-FP
tccgagggcaaaggaataatgacggggcgcgtctggaacaatca

SEQ ID NO: 10 - DNA, 39 bp, Artificial
  primer sequence of WPRE-RP
caacacaggcgagcagccatggaaaggacgtcagcttcc

SEQ ID NO: 11 - DNA, 43 bp, Artificial
  primer sequence of EF1a-FP
ggtagtctcaagctggccggcctgctctggtgcctggcctcgc

SEQ ID NO: 12 - DNA, 64 bp, Artificial
  primer sequence of EF1a-RP
gagtagtgagaaattcgtggcaccagatcctctagactgcagatcggcaccgggcttgcgggtc

SEQ ID NO: 13 - DNA, 77 bp, Artificial
  primer sequence of p2A-EGFP-FP
gccacgaatttctcactactcaagcaggccggtgatgtcgaggaaaaccctggtcctgtgagcaagggcgaggagct

SEQ ID NO: 14 - DNA, 41 bp, Artificial
  primer sequence of p2A-EGFP-RP
gattgtcgacttaacgcgtttacttgtacagctcgtccatg

SEQ ID NO: 15 - DNA, 42 bp, Artificial
  primer sequence of WPRE-LTR-FP
catggacgagctgtacaagtaaacgcgttaagtcgacaatca

SEQ ID NO: 16 - DNA, 40 bp, Artificial
  primer sequence of WPRE-LTR-RP
aaaaaaattagtcagccatggggcggagaatgggcggaac

SEQ ID NO: 17 - DNA, 55 bp, Artificial
  primer sequence of Oligo-Amplifying-FP
ttgaaagtatttcgatttcttggctttatatatcttgtggaaaggacgaaacacc

SEQ ID NO: 18 - DNA, 57 bp, Artificial
  primer sequence of Oligo-Amplifying-RP
gagtaagctgaccgctgaagtacaagtggtagagtagagatctagttacgccaagct

SEQ ID NO: 19 - DNA, 119 bp, Artificial
  primer sequence of Improved scaffods with UMI
  misc_feature (93)..(100): n is a, c, g, or t
gtttcagagctatgctggaaacagcatagcaagttgaaataaggctagtccgttatcaacttgaaaaagtggcaccgagtcggtgcttttttnnnnnnnntttgggagacgcgatcgat

SEQ ID NO: 20 - DNA, 49 bp, Artificial
  primer sequence of Scaffold-Amplifying FP
caagcttggtaccgagctcgttttcgtctctgtttcagagctatgctgg

SEQ ID NO: 21 - DNA, 39 bp, Artificial
  primer sequence of Scaffold-Amplifying-RP
tatagggcgaattgggccctatcgatcgcgtctcccaaa

SEQ ID NO: 22 - DNA, 56 bp, Artificial
  primer sequence of 1st Deep sequencing FP-A
acactctttccctacacgacgctcttccgatctcttgaaaaagtggcaccgagtcg

SEQ ID NO: 23 - DNA, 57 bp, Artificial
  primer sequence of 1st Deep sequencing FP-B
acactctttccctacacgacgctcttccgatcttcttgaaaaagtggcaccgagtcg

SEQ ID NO: 24 - DNA, 58 bp, Artificial
  primer sequence of 1st Deep sequencing FP-C
acactctttccctacacgacgctcttccgatctcgcttgaaaaagtggcaccgagtcg

SEQ ID NO: 25 - DNA, 61 bp, Artificial
  primer sequence of 1st Deep sequencing RP-A
gtgactggagttcagacgtgtgctcttccgatctttaagtcgagtaagctgaccgctgaag

SEQ ID NO: 26 - DNA, 62 bp, Artificial
  primer sequence of 1st Deep sequencing RP-B
gtgactggagttcagacgtgtgctcttccgatctattaagtcgagtaagctgaccgctgaag

SEQ ID NO: 27 - DNA, 63 bp, Artificial
  primer sequence of 1st Deep sequencing RP-C
gtgactggagttcagacgtgtgctcttccgatcttattaagtcgagtaagctgaccgctgaag

SEQ ID NO: 28 - DNA, 29 bp, Artificial
  front part of 2nd Illuimina indexing FP
aatgatacggcgaccaccgagatctacac

SEQ ID NO: 29 - DNA, 20 bp, Artificial
  end part of 2nd Illuimina indexing FP
acactctttccctacacgac

SEQ ID NO: 30 - DNA, 24 bp, Artificial
  front part of 2nd Illuimina indexing RP
caagcagaagacggcatacgagat

SEQ ID NO: 31 - DNA, 21 bp, Artificial
  end part of 2nd Illuimina indexing RP
gtgactggagttcagacgtgt

SEQ ID NO: 32 - DNA, 19 bp, Artificial
  GN19 guide sequence of Cg.TP53_p.T155I,p.T155T
ggcacccgcgtccgcgcca

SEQ ID NO: 33 - DNA, 53 bp, Artificial
  Adaptor_FP of Cg.TP53_p.T155I,p.T155T
acactctttccctacacgacgctcttccgatctttgcccagggtccccaggcc

SEQ ID NO: 34 - DNA, 53 bp, Artificial
  Adaptor_RP of Cg.TP53_p.T155I,p.T155T
gtgactggagttcagacgtgtgctcttccgatcttctccagccccagctgctc

SEQ ID NO: 35 - DNA, 19 bp, Artificial
  GN19 guide sequence of Cg.TP53_p.Q100*
cttcccagaaaacctacca

SEQ ID NO: 36 - DNA, 53 bp, Artificial
  Adaptor_FP of Cg.TP53_p.Q100*
acactctttccctacacgacgctcttccgatctcccctgtcatcttctgtccc

SEQ ID NO: 37 - DNA, 54 bp, Artificial
  Adaptor_RP of Cg.TP53_p.Q100*
gtgactggagttcagacgtgtgctcttccgatctaatgcagggggatacggcca

SEQ ID NO: 38 - DNA, 19 bp, Artificial
  GN19 guide sequence of Cg.CDC23_p.T381M
aacacgtctgctgctatcc

SEQ ID NO: 39 - DNA, 20 bp, Artificial
  Amplifying_FP of Cg.CDC23_p.T381M
tgggtgttttgccaggactt

SEQ ID NO: 40 - DNA, 20 bp, Artificial
  Amplifying_RP of Cg.CDC23_p.T381M
caagtgaccaggcttaccga

SEQ ID NO: 41 - DNA, 53 bp, Artificial
  Adaptor_FP of Cg.CDC23_p.T381M
acactctttccctacacgacgctcttccgatctggtgcctggacactaatggg

SEQ ID NO: 42 - DNA, 54 bp, Artificial
  Adaptor_RP of Cg.CDC23_p.T381M
gtgactggagttcagacgtgtgctcttccgatctctgcccgaggccataccaag

SEQ ID NO: 43 - DNA, 19 bp, Artificial
  GN19 guide sequence of Cg.PTPN14_p.Q110*
gcttcagcaagaggccaca

SEQ ID NO: 44 - DNA, 20 bp, Artificial
  Amplifying_FP of Cg.PTPN14_p.Q110*
cttacctcacatgggcgctt

SEQ ID NO: 45 - DNA, 20 bp, Artificial
  Amplifying_RP of Cg.PTPN14_p.Q110*
ttatatggcacacaggggga

SEQ ID NO: 46 - DNA, 53 bp, Artificial
  Adaptor_FP of Cg.PTPN14_p.Q110*
acactctttccctacacgacgctcttccgatctcaagagccagcaagcacgat

SEQ ID NO: 47 - DNA, 54 bp, Artificial
  Adaptor_RP of Cg.PTPN14_p.Q110*
gtgactggagttcagacgtgtgctcttccgatctggcacacagggggaaaatgc

SEQ ID NO: 48 - DNA, 19 bp, Artificial
  GN19 guide sequence of Cg.TP53_p.Q192*
cctcagcatcttatccgag

SEQ ID NO: 49 - DNA, 53 bp, Artificial; identical to SEQ ID NO: 33
  Adaptor_FP of Cg.TP53_p.Q192*

SEQ ID NO: 50 - DNA, 54 bp, Artificial
  Adaptor_RP of Cg.TP53_p.Q192*
gtgactggagttcagacgtgtgctcttccgatctagggccactgacaaccaccc

SEQ ID NO: 51 - DNA, 19 bp, Artificial
  GN19 guide sequence of Cg.POLR1C_p.A6V
tcaggcggtggaggaaatg

SEQ ID NO: 52 - DNA, 18 bp, Artificial
  Amplifying_FP of Cg.POLR1C_p.A6V
tgggatcggccggaacac

SEQ ID NO: 53 - DNA, 21 bp, Artificial
  Amplifying_RP of Cg.POLR1C_p.A6V
cccaggcatcatcataaccgg

SEQ ID NO: 54 - DNA, 60 bp, Artificial
  Adaptor_FP of Cg.POLR1C_p.A6V
acactctttccctacacgacgctcttccgatctctcgcgatatttaagattccaggaggc

SEQ ID NO: 55 - DNA, 58 bp, Artificial
  Adaptor_RP of Cg.POLR1C_p.A6V
gtgactggagttcagacgtgtgctcttccgatctacgaatttgtccacgaagggacag

SEQ ID NO: 56 - DNA, 19 bp, Artificial
  GN19 guide sequence of Cg.MMS22L_p.R661*
atgtcgagaatctgaactt

SEQ ID NO: 57 - DNA, 23 bp, Artificial
  Amplifying_FP of Cg.MMS22L_p.R661*
gagagccctcatttggaagggtc

SEQ ID NO: 58 - DNA, 32 bp, Artificial
  Amplifying_RP of Cg.MMS22L_p.R661*
cggatatattcaacctgtactgatttatgccc

SEQ ID NO: 59 - DNA, 59 bp, Artificial
  Adaptor_FP of Cg.MMS22L_p.R661*
acactctttccctacacgacgctcttccgatctggtacagagacagactatctggaccc

SEQ ID NO: 60 - DNA, 59 bp, Artificial
  Adaptor_RP of Cg.MMS22L_p.R661*
gtgactggagttcagacgtgtgctcttccgatctctgaaaaccatacctgattctggcc

SEQ ID NO: 61 - DNA, 19 bp, Artificial
  GN19 guide sequence of Cg.POLR2B_p.P714L
ttcctgatcataaccaggt

SEQ ID NO: 62 - DNA, 22 bp, Artificial
  Amplifying_FP of Cg.POLR2B_p.P714L
ttagttggcaggatcttgtggc

SEQ ID NO: 63 - DNA, 22 bp, Artificial
  Amplifying_RP of Cg.POLR2B_p.P714L
catacctgctggcagctctcta

SEQ ID NO: 64 - DNA, 53 bp, Artificial
  Adaptor_FP of Cg.POLR2B_p.P714L
acactctttccctacacgacgctcttccgatctgcaggatcttgtggccagtg

SEQ ID NO: 65 - DNA, 64 bp, Artificial
  Adaptor_RP of Cg.POLR2B_p.P714L
gtgactggagttcagacgtgtgctcttccgatctccctccagataagactacagagttactttc

SEQ ID NO: 66 - DNA, 19 bp, Artificial
  GN19 guide sequence of Cg.POLG_p.Q1029*
ggtccagagagaaactgca

SEQ ID NO: 67 - DNA, 27 bp, Artificial
  Amplifying_FP of Cg.POLG_p.Q1029*
tgatatgtgaacattccttgccaaggc

SEQ ID NO: 68 - DNA, 25 bp, Artificial
  Amplifying_RP of Cg.POLG_p.Q1029*
tgctccaaaggtagcaagatacctc

SEQ ID NO: 69 - DNA, 52 bp, Artificial
  Adaptor_FP of Cg.POLG_p.Q1029*
acactctttccctacacgacgctcttccgatctccccaggtatcggctgtcg

SEQ ID NO: 70 - DNA, 55 bp, Artificial
  Adaptor_RP of Cg.POLG_p.Q1029*
gtgactggagttcagacgtgtgctcttccgatctagcatccaagctcttctgggg

SEQ ID NO: 71 - DNA, 19 bp, Artificial
  GN19 guide sequence of Cg.ACOX3_p.Q145*
attcaaaagatcttcagga

SEQ ID NO: 72 - DNA, 20 bp, Artificial
  Amplifying_FP of Cg.ACOX3_p.Q145*
actcttcttacctgccccct

SEQ ID NO: 73 - DNA, 21 bp, Artificial
  Amplifying_RP of Cg.ACOX3_p.Q145*
caagaagagcataagccccct

SEQ ID NO: 74 - DNA, 53 bp, Artificial
  Adaptor_FP of Cg.ACOX3_p.Q145*
acactctttccctacacgacgctcttccgatctttcttacctgccccctgttg

SEQ ID NO: 75 - DNA, 55 bp, Artificial
  Adaptor_RP of Cg.ACOX3_p.Q145*
gtgactggagttcagacgtgtgctcttccgatctagaagagcataagccccctgg

SEQ ID NO: 76 - DNA, 19 bp, Artificial
  GN19 guide sequence of Cg.KMT2C_p.R1906*
cccctcgaccacctcctgt

SEQ ID NO: 77 - DNA, 20 bp, Artificial
  Amplifying_FP of Cg.KMT2C_p.R1906*
agacttctcagccaccctca

SEQ ID NO: 78 - DNA, 20 bp, Artificial
  Amplifying_RP of Cg.KMT2C_p.R1906*
caggggatggcctatttgct

SEQ ID NO: 79 - DNA, 53 bp, Artificial
  Adaptor_FP of Cg.KMT2C_p.R1906*
acactctttccctacacgacgctcttccgatctgacttctcagccaccctcac

SEQ ID NO: 80 - DNA, 54 bp, Artificial
  Adaptor_RP of Cg.KMT2C_p.R1906*
gtgactggagttcagacgtgtgctcttccgatctttccactggtgcagcagaat

SEQ ID NO: 81 - DNA, 19 bp, Artificial
  GN19 guide sequence of Ag.TP53_p.R280G
gggagagaccggcgcacag

SEQ ID NO: 82 - DNA, 53 bp, Artificial
  Adaptor_FP of Ag.TP53_p.R280G
acactctttccctacacgacgctcttccgatctgggacaggtaggacctgatt

SEQ ID NO: 83 - DNA, 54 bp, Artificial
  Adaptor_RP of Ag.TP53_p.R280G
gtgactggagttcagacgtgtgctcttccgatctaccgcttcttgtcctgcttg

SEQ ID NO: 84 - DNA, 19 bp, Artificial
  GN19 guide sequence of Ag.TP53_p.N239D
tgtgtaacagttcctgcat

SEQ ID NO: 85 - DNA, 22 bp, Artificial
  Amplifying_FP of Ag.TP53_p.N239D
gaagcttacagaggctaagggc

SEQ ID NO: 86 - DNA, 20 bp, Artificial
  Amplifying_RP of Ag.TP53_p.N239D
gtaaggagattccccgccgg

SEQ ID NO: 87 - DNA, 52 bp, Artificial
  Adaptor_FP of Ag.TP53_p.N239D
acactctttccctacacgacgctcttccgatctctgcttgccacaggtctcc

SEQ ID NO: 88 - DNA, 53 bp, Artificial
  Adaptor_RP of Ag.TP53_p.N239D
gtgactggagttcagacgtgtgctcttccgatctcagtgtgcagggtggcaag

SEQ ID NO: 89 - DNA, 19 bp, Artificial
  GN19 guide sequence of Ag.TP53_p.K120E
gccaagtctgtgacttgca

SEQ ID NO: 90 - DNA, 53 bp, Artificial; identical to SEQ ID NO: 36
  Adaptor_FP of Ag.TP53_p.K120E

SEQ ID NO: 91 - DNA, 54 bp, Artificial; identical to SEQ ID NO: 37
  Adaptor_RP of Ag.TP53_p.K120E

SEQ ID NO: 92 - DNA, 19 bp, Artificial
  GN19 guide sequence of Ag.TP53_p.K351E
actcaaggatgcccaggct

SEQ ID NO: 93 - DNA, 21 bp, Artificial
  Amplifying_FP of Ag.TP53_p.K351E
tgcatgttgcttttgtaccgt

SEQ ID NO: 94 - DNA, 20 bp, Artificial
  Amplifying_RP of Ag.TP53_p.K351E
ctgggacccaatgagatggg

SEQ ID NO: 95 - DNA, 53 bp, Artificial
  Adaptor_FP of Ag.TP53_p.K351E
acactctttccctacacgacgctcttccgatctcttctccccctcctctgttg

SEQ ID NO: 96 - DNA, 54 bp, Artificial
  Adaptor_RP of Ag.TP53_p.K351E
gtgactggagttcagacgtgtgctcttccgatctgaaggcaggggagtagggcc

SEQ ID NO: 97 - DNA, 19 bp, Artificial
  GN19 guide sequence of Ag.TP53_p.T125A
tgcacggtcagttgccctg

SEQ ID NO: 98 - DNA, 53 bp, Artificial; identical to SEQ ID NO: 36
  Adaptor_FP of Ag.TP53_p.T125A

SEQ ID NO: 99 - DNA, 54 bp, Artificial; identical to SEQ ID NO: 37
  Adaptor_RP of Ag.TP53_p.T125A

SEQ ID NO: 100 - DNA, 19 bp, Artificial
  GN19 guide sequence of Ag.CTCF_p.H312R
atcaccttaacacacacac

SEQ ID NO: 101 - DNA, 20 bp, Artificial
  Amplifying_FP of Ag.CTCF_p.H312R
cagttacacgtgtccacggc

SEQ ID NO: 102 - DNA, 22 bp, Artificial
  Amplifying_RP of Ag.CTCF_p.H312R
ccaggcatctattgcctgagac

SEQ ID NO: 103 - DNA, 53 bp, Artificial
  Adaptor_FP of Ag.CTCF_p.H312R
acactctttccctacacgacgctcttccgatctcagttacacgtgtccacggc

SEQ ID NO: 104 - DNA, 59 bp, Artificial
  Adaptor_RP of Ag.CTCF_p.H312R
gtgactggagttcagacgtgtgctcttccgatctcttcctttaaattcccgctggagtc

SEQ ID NO: 105 - DNA, 19 bp, Artificial
  GN19 guide sequence of Ag.SRSF1_p.D139G
aggatcacatgcgtgaagc

SEQ ID NO: 106 - DNA, 20 bp, Artificial
  Amplifying_FP of Ag.SRSF1_p.D139G
cgaggattgctgctgtggtg

SEQ ID NO: 107 - DNA, 25 bp, Artificial
  Amplifying_RP of Ag.SRSF1_p.D139G
caaccttgcctgaatccttaccttg

SEQ ID NO: 108 - DNA, 62 bp, Artificial
  Adaptor_FP of Ag.SRSF1_p.D139G
acactctttccctacacgacgctcttccgatctccagctctctttacctggtatcacttaag

SEQ ID NO: 109 - DNA, 56 bp, Artificial
  Adaptor_RP of Ag.SRSF1_p.D139G
gtgactggagttcagacgtgtgctcttccgatctcgtacaaactccacgacaccag

SEQ ID NO: 110 - DNA, 19 bp, Artificial
  GN19 guide sequence of Ag.POLE_p.Y1889C
agtacatcaccagcaggtg

SEQ ID NO: 111 - DNA, 20 bp, Artificial
  Amplifying_FP of Ag.POLE_p.Y1889C
ggaccctcagctcttttccc

SEQ ID NO: 112 - DNA, 24 bp, Artificial
  Amplifying_RP of Ag.POLE_p.Y1889C
cttcctgaacttgcccaactcaag

SEQ ID NO: 113 - DNA, 52 bp, Artificial
  Adaptor_FP of Ag.POLE_p.Y1889C
acactctttccctacacgacgctcttccgatcttgggtgccctctggctctc

SEQ ID NO: 114 - DNA, 57 bp, Artificial
  Adaptor_RP of Ag.POLE_p.Y1889C
gtgactggagttcagacgtgtgctcttccgatctgcacctcagggggtcattttagc

SEQ ID NO: 115 - DNA, 19 bp, Artificial
  GN19 guide sequence of Ag.ACTL6A_p.T405A
gggtacctttcaacagatg

SEQ ID NO: 116 - DNA, 21 bp, Artificial
  Amplifying_FP of Ag.ACTL6A_p.T405A
aggtgggagcatcccttgaac

SEQ ID NO: 117 - DNA, 25 bp, Artificial
  Amplifying_RP of Ag.ACTL6A_p.T405A
agcctaaggtaaaaagcataggcag

SEQ ID NO: 118 - DNA, 57 bp, Artificial
  Adaptor_FP of Ag.ACTL6A_p.T405A
acactctttccctacacgacgctcttccgatcttggctgacagagcaagaccttctc

SEQ ID NO: 119 - DNA, 57 bp, Artificial
  Adaptor_RP of Ag.ACTL6A_p.T405A
gtgactggagttcagacgtgtgctcttccgatctggaaggtagaagcttgggaactc

(19) United States
(12) Patent Application Publication
(10) Pub. No.: US 2022/0392569 A1
(43) Pub. Date: Dec. 8, 2022

(54) METHOD FOR EVALUATING THE FUNCTION OF CANCER MUTATIONS THROUGH BASE EDITOR AND EVALUATION SYSTEM USING THE SAME

(71) Applicant: Industry-Academic Cooperation Foundation, Yonsei University, Seoul (KR)

(72) Inventors: Hyongbum Henry KIM, Seoul (KR); Younggwang KIM, Seoul (KR); Seungho LEE, Seoul (KR)

(73) Assignee: INDUSTRY-ACADEMIC COOPERATION FOUNDATION, YONSEI UNIVERSITY, Seoul (KR)

(21) Appl. No.: 17/825,394

(22) Filed: May 26, 2022

(30) Foreign Application Priority Data
May 26, 2021 (KR) 10-2021-0067906
May 25, 2022 (KR) 10-2022-0064208

(51) Int. Cl. G16B 20/00; C12Q 1/6886; C12N 15/10; G16B 40/20

(52) U.S. Cl. CPC G16B 20/00; C12Q 1/6886; C12N 15/1082; G16B 40/20; C12Q 1/6869

(57) ABSTRACT

The disclosure relates to a method of evaluating functions of cancer mutations using base editors and guide RNAs, an evaluation system for mutations, and a computer-readable recording medium in which is recorded a program for executing the method by a computer.

### CROSS-REFERENCE TO RELATED APPLICATION

This application claims the benefit of Korean Patent Application No. 10-2021-0067906, filed on May 26, 2021, and Korean Patent Application No. 10-2022-0064208, filed on May 25, 2022, in the Korean Intellectual Property Office, the disclosure of which is incorporated herein in its entirety by reference.

### BACKGROUND

### 1. Field

The disclosure relates to a method of evaluating functions of cancer mutations using base editors and guide RNAs, an evaluation system for mutations, and a computer-readable recording medium in which is recorded a program for executing the method by a computer.

### 2. Description of the Related Art

Cancer is a disease caused by mutations in the DNA sequence of cells. Numerous mutations have been found in cancer due to the development of high-throughput genetic analysis technology (sequencing technology), but identifying important mutations related to cancer development and malignancy has been difficult, and there has been a challenge in utilizing the mutation information found in a patient.

In order to know the effect of a specific mutation on cancer development, in the past, a statistical method of finding a mutation frequently observed in cancers was used, however, this method has a limitation in that it cannot determine the causal relationship between mutations and tumorigenesis and proliferation. In addition, it was impossible to induce various mutations in large quantities throughout the genome with existing techniques.

Nonetheless, recently, base editors capable of editing the genome without cutting DNA have been developed, such as adenine base editors (ABEs) and cytosine base editors (CBEs). Cytosine base editors are constructed by fusing a naturally-derived cytosine deaminase to dCas9 or nCas9, and are able to convert cytosine to thymine without cutting the gene or inserting an additional donor DNA. On the other hand, adenine base editors are constructed by fusing an artificially modified adenosine deaminase with a Cas9 variant, and are known to be capable of converting adenine to guanine. These base editors are attracting attention as a tool that will advance the research and treatment of refractory genetic diseases, in addition, genetic editing techniques and research using the same are in progress.

In particular, interest is increasing in a method of evaluating the effect of single-nucleotide cancer mutations observed in human cancers on cell proliferation by using base editors, which are the next-generation gene editing technology.

### SUMMARY

An aspect provides a method of evaluating a function of a cancer mutation by using guide RNAs and base editors.

An aspect provides an evaluation system for cancer mutations by using guide RNAs and base editors.

Another aspect provides a computer-readable recording medium in which a program is recorded for executing the method by a computer.

The method according to an aspect identifies the relationship between cancer mutations and cell proliferation by directly introducing about 30,000 to 100,000 single nucleotide cancer mutations observed in human cancers into cells, and this allows classification of the function of a mutation based on its effect on cell proliferation. In addition, according to the method and the system of an aspect, the function of about 100,000 cancer mutations may be evaluated at once, and the result of base editing through a base editing gene may be accurately identified at the level of a single nucleotide, and thus, mutations matching the mutation type extracted from the cancer database may be accurately extracted, and mutations resistant to anticancer drugs may be effectively discovered.

### BRIEF DESCRIPTION OF DRAWINGS

These and/or other aspects will become apparent and more readily appreciated from the following description of the embodiments, taken in conjunction with the accompanying drawings in which:

FIG. 1 schematically shows maps of lentiviral vectors used for the expression of rtTA (pLVX-EF1a-rtTA-neomycin), CBE (TRE3G-AncBE4max-PGK-hygromycin) and ABE (TRE3G-ABEmax-PGK-hygromycin). These vectors were used to generate P-C and P-A cells, rtTA: reverse tetracycline controlled transactivator; Anc689APOBEC: codon- optimized ancestral APOBEC1 (AncBE4max); TadA: tRNA adenosine deaminase; bis-bpNLS: biparticle nuclear localization signal at both the N- and C-termini; TRE3G: tetracycline response element 3G promoter.

FIG. 2 schematically illustrates a method of generating a lentiviral library of sgRNA-encoding sequence and target sequence pairs having a unique molecular identifier (UMI). Plasmid library 1 was generated by synthesizing an oligonucleotide containing a 20-nt guide sequence and the corresponding target sequence and cloning the same into a pLenti-gRNA-puro vector, and to generate plasmid library 2, the plasmid was then digested with BsmBI restriction enzyme and ligated with a fragment containing the sgRNA scaffold sequence and UMI. The lentiviral library generated from plasmid library 2 was transduced into cells that express a cytosine base editor (CBE) or an adenine base editor (ABE) in a doxycycline-induced manner.

FIG. 3A schematically illustrates a method of designing small-scale libraries C1, C2 and A1; FIG. 3B schematically shows a method of designing small-scale libraries C3 and A2. In addition, FIG. 3C shows the correlation between nonsynonymous base editing efficiencies at the integrated target sequences of biological replicates. The base editing efficiencies of FIG. 3C were measured 10 days after the initial transduction of each library into P-C or P-A cells; Only sgRNAs with more than 100 raw read counts for each replicate were included; Pearson correlation coefficients (r) are shown, and the number of sgRNAs n=3,181 (Library C1), 3,063 (Library C2) and 1,520 (Library A1).

FIG. 4A shows the base editing efficiency measured at each position of the marked region for the target nucleotide C in the surrogate target sequence; and FIG. 4B identifies the base editing efficiency measured at each position of the marked region for the target nucleotide A in the surrogate target sequence. Position 1 refers to the 5′-end of the target sequence and position 20 refers to a position immediately upstream of the NGG PAM. The number of sequence (n) to be analyzed is as follows: n=5,865 (position-4), 5,393 (position-3), 5,782 (position-2), 5,815 (position-1), 5,292 (position 1), 5,614 (position 2), 5,697, 6,394, 10,586, 9,382, 8,837, 5,421, 6,130, 5,339, 5,541, 5,796, 5,058, 5,723, 5,955, 5,348, 5,779, 5,437, 4,884, 5,502 (position 20) in FIG. 4A; n=19,475 (position-4), 20,753 (position-3), 20,110 (position-2), 19,425 (position-1), 19,984 (position 1), 20,004 (position 2), 17,873, 24,870, 35,421, 33,186, 32,807, 19,895, 19,195, 20,227, 19,549, 18,986, 20,367, 18,793, 18,361, 20,478, 19,605, 20,975, 21,542, 22,952 (position 20) in FIG. 4B.

FIG. 5 illustrates a functional classification method in order. Specifically, (Step 1) sgRNAs harboring more than 50 unique molecular identifiers (UMIs) were used as inputs for MAGECK analysis. (Step 2) sgRNAs associated with a nonsynonymous editing efficiency of less than 60% in the integrated target sequence were eliminated. (Step 3) sgRNAs were grouped depending on their normalized log fold changes (nLFCs) and P-values obtained from MAGECK-UMI analyses. The cutoff value was determined by the distribution of the non-targeting controls in each library. (Step 4) For the outgrowing and depleting groups, UMI CPM (counts per million) LFCs were further considered to prevent false classification into outgrowing and depleting groups. The number of sgRNAs and mutant proteins classified in each group are shown in the chart (integrated results based on libraries C, C1, C2, C3, A, A1, A2, and dA are shown).

FIG. 6 schematically shows a map of the lentiviral vector containing the library of sgRNA-encoding sequence and surrogate target sequence pairs. Here, UMI is a 8-nt unique molecular identifier.

FIG. 7 schematically illustrates CBE- and ABE-mediated high-throughput evaluations of variants.

FIG. 8 illustrates correlations between nonsynonymous base editing efficiencies at the integrated target sequences of biological replicates. The color of each dot was determined by the number of neighboring dots (i.e., dots within a distance that is three times the radius of the dot), and Pearson correlation coefficients (r) are shown.

FIG. 9 shows the distribution of median normalized log fold changes (LFCs) of 190 sgRNAs targeting essential genes depending on the nonsynonymous base editing efficiencies determined at the integrated target sequences in library C2: NT, non-targeting sgRNAs; The number of sgRNAs n=99 (NT), 13 (<20%), 17 (20%˜40%), 31 (40%˜60%), 129 (>60%). (in comparison with NT; two-sided Student's t test; NS, not significant, *P=6.1×10⁻⁷, **P=2.3×10⁻²¹).

FIG. 10 shows volcano plots of nLFCs for libraries C and A, identifying negative logarithm of robust rank aggregation (RRA) P-values of sgRNAs, and showing functional classifications of sgRNAs, wherein non-targeting sgRNAs are shown in black (dark dots).

FIG. 11 shows correlations between nonsynonymous base editing efficiencies at the integrated target sequences of libraries C and A and small libraries C1, C2, and A1.

FIG. 12 shows volcano plots of nLFCs for small libraries C1, C2, and A1, indicating negative logarithm of RRA P-values of sgRNAs.

FIG. 13 is heat maps showing the correlations between functional classifications made by using libraries C and A and small libraries C1, C2, and A1; the color intensity was determined by the relative number of variants within each cell in each row. D, Depleting, LD, Likely depleting, LND, Likely neutral (Possibly depleting), N, Neutral, LNO, Likely neutral (Possibly outgrowing), LO (Likely outgrowing), O (Outgrowing).

FIG. 14 shows volcano plots of nLFCs for libraries C3, A2 and dA and negative logarithm of RRA P-values of sgRNAs.

FIG. 15 is diagrams showing correlation of individual validation of sgRNAs and their associated base-edited variants and the high-throughput functional classification method, and FIG. 15A is a schematic of experiments for validating the function of each sgRNA and variants, and competitive proliferation (top) and allele frequency tracking (bottom) assays are shown. FIG. 15B shows correlation between frequencies of base-edited outcome sequences induced by base editing at endogenous target sites in individual validation experiments and corresponding integrated target sequences in the high-throughput experiments; Spearman correlation (R) and Pearson correlation (r) coefficients are shown, base-editing outcomes with frequencies higher than 1% are included; and the number of base editing outcome sequences n=57. FIG. 15C is a diagram showing correlation of phenotypes caused by sgRNA-induced base editing determined by individual allele frequency tracking experiments and high-throughput experiments; the number of sgRNAs n=20. FIG. 15D is a diagram identifying correlation of phenotypes caused by sgRNA-induced base editing determined by individual competitive proliferation assays and high-throughput experiments; the number of sgRNAs n=24; statistical significances determined by comparison with the neutral/likely neutral group are shown (two-sided Mann-Whitney U test).

FIG. 16 is a diagram illustrating an experiment identifying mutants having resistance to the EGFR tyrosine kinase inhibitor afatinib through a base-editor-mediated mutation, FIG. 16A is a schematic of CBE-mediated high-throughput evaluations of variants conferring resistance to the EGFR tyrosine kinase inhibitor afatinib, FIG. 16B is volcano plots of nLFCs and shows negative logarithm of RRA P-values of sgRNAs in the experiment identifying mutants having resistance to the EGFR tyrosine kinase inhibitor afatinib through a base-editor-mediated mutation, and FIG. 16C is a diagram showing the numbers of sgRNAs and mutant proteins classified in each group.

FIG. 17 is diagrams identifying correlation of genes related to sgRNAs and protein variants, FIG. 17A (top) shows notable gene groups related to the outgrowing phenotype including the cancer gene census (CGC), and identifies functional classification of the same, FIG. 17A (bottom) is a diagram showing the results of the same analysis performed by using 29,060 functionally classified protein variants; the left side of FIG. 17B is a diagram showing the results of confirming a notable gene group related to the depleting phenotype, and the right side of FIG. 17B is a diagram showing the results of analysis using 29,060 functionally classified protein variants instead of sgRNAs in the same manner.

### DETAILED DESCRIPTION

An aspect provides a method of evaluating a function of a cancer mutation using guide RNAs and base editors.

The method of evaluating a function of a cancer mutation includes: generating a cell library including a nucleotide sequence encoding a guide RNA, a unique molecular identifier (UMI) nucleotide sequence, and an oligonucleotide including a target nucleotide sequence targeted by the guide RNA;

transducing the cell library into cells expressing the base editors and culturing the cells;

harvesting the transduced cells after culturing, performing deep sequencing, and measuring the data of the base editing efficiency and the frequency level of protein mutations due to base editing; and

analyzing the measured data to evaluate the function of the mutation introduced into the cell library.

The present inventors constructed cell libraries, in which each mutation was induced, by using a guide RNA coding sequence capable of inducing 100,000 mutations and a corresponding target sequence through a high-throughput experiment, transduced the same into cells expressing the base editors, and identified the efficiency of the large scale mutations through the next-generation sequencing technology of the target nucleotide sequence. In addition, it was found that about 30,000 base editing results of the base editors may be accurately identified at the level of a single nucleotide, and a large amount of data analysis is confirmed to be possible.

The term “base editor” (BE) refers to a means of editing a single base, and more specifically, a base editor may be constructed by fusing a cytosine deaminase or an adenine deaminase to the N-terminus of a Cas9 nickase. The base editor may include cytosine base editors (CBEs) and adenine base editors (ABEs). The BE does not cause double-strand breaks, ABE converts adenine to guanine at a specific site, and CBE converts cytosine to thymine at a specific site. In the present specification, the term “base editor” may be used interchangeably with “base editing genetic scissors”, “genetic scissors”, or “base converter”.

The term “guide RNA” refers to a polynucleotide that recognizes a target nucleic acid through genome editing and cuts, inserts, or connects the target nucleic acid. The guide RNA may include a sequence complementary to the target nucleic acid. The guide RNA may include a polynucleotide complementary to a nucleotide sequence of 2 to 24 consecutive nucleotides (for example, around 20 nt) (hereinafter referred to as ‘nt’) in the 5′-direction or 3′-direction of the PAM, in the target nucleic acid. The length of the guide RNA may be 10 nt to 100 nt, 10 nt to 90 nt, 10 nt to 80 nt, 10 nt to 70 nt, 10 nt to 60 nt, 10 nt to 50 nt, 15 nt to 50 nt, or 20 nt to 50 nt. The guide RNA may be, for example, a single guide RNA (sgRNA).

The “base conversion efficiency” means genetic editing efficiency by a base editor. The base conversion efficiency may be calculated as a rate at which an editing occurs without unintentional mutation in the target sequence when gene editing is performed by using a base editor, wherein the editing is induced by a base editor and a guide RNA. The efficiency of the base conversion may be expressed as a percentage.

“Target sequence” refers to a target nucleotide sequence an sgRNA targets. The target sequence may be a sequence expected to be targeted by an sgRNA. The target sequence may be a partial sequence of known genomic sequences, or a sequence arbitrarily designed by a person skilled in the art to be analyzed by using the system of the disclosure.

“Oligonucleotide” refers to a substance in which several to hundreds of nucleotides are linked by a phosphodiester bond. The length of the oligonucleotide may be 100 nts to 300 nts, 100 nts to 250 nts, or 100 nts to 200 nts, but is not limited thereto, and those skilled in the art may appropriately adjust the length.

The oligonucleotide may further include a barcode sequence. Accordingly, the oligonucleotide may include a sequence encoding an sgRNA, a unique molecular identifier (UMI) nucleotide sequence, a barcode sequence, and a target sequence targeted by the sgRNA. The number of barcode sequences may be one, two, or more. The barcode sequence may be appropriately designed by those skilled in the art according to the purpose. For example, the barcode sequence may be such that enables each sgRNA and a corresponding target sequence pair to be identified after deep sequencing is performed.

“Library” means a pool or population containing two or more types of substances of the same kind with different characteristics. Accordingly, an oligonucleotide library may be a pool including two or more oligonucleotides having different nucleotide sequences, for example, sgRNAs, and/or two or more oligonucleotides having different target sequences. In addition, the cell library may be a pool of two or more types of cells having different characteristics, for example, cells having different oligonucleotides contained in the cells.

“Vector” may refer to a medium that allows the oligonucleotide to be delivered into a cell. Specifically, a vector may include a oligonucleotide including each sequence encoding a sgRNA and a target sequence. The vector may be a viral vector or a plasmid vector, but is not limited thereto. The viral vector may be a lentiviral vector or a retroviral vector, but is not limited thereto. The vector may contain necessary regulatory elements operably linked to the insert, that is, the oligonucleotide, so that the oligonucleotide may be expressed when the vectors are present in the cells of the subject. The vector may be prepared and purified by using standard recombinant DNA techniques. The type of the vector is not particularly limited as long as it may act in target cells such as prokaryotic cells and eukaryotic cells. A vector may include a promoter, an initiation codon, and a stop codon terminator. In addition, DNA coding a signal peptide, and/or an enhancer sequence, and/or a 5′ or 3′ untranslated region, and/or a selectable marker region, and/or a replication unit may also be appropriately included.

A method of delivering the vector to a cell to preparing a library may be accomplished using various methods known in the art. Various methods known in the art may be performed, for example, calcium phosphate-DNA co-precipitation method, diethylaminoethyl (DEAE)-dextran-mediated transfection method, polybrene-mediated transfection method, electroporation method, microinjection method, liposome fusion method, lipofectamine and protoplast fusion method, and the like. Furthermore, when a viral vector is used, infection of the virus particles may be used as a means to deliver the object, that is, a vector to the cells. In addition, the vector may be introduced into the cell by gene gun bombardment and the like. The introduced vector may exist in the cell as a vector itself or may be integrated into the chromosome, but its manner of existence is not limited thereto.

The type of cell into which the vector may be introduced may be appropriately selected by those skilled in the art according to the type of the vector and/or the type of the target cell, but for example, bacterial cells such as Escherichia coli, Streptomyces, and Salmonella typhimurium; yeast cells; fungal cells such as Pichia pastoris; insect cells such as Drosophila and Spodoptera Sf9 cells; animal cells such as Chinese hamster ovary cells (CHO), SP2/0 (mouse myeloma), human lymphoblastoid, COS, NSO (mouse myeloma), 293T, Bow melanoma cells, HT-1080, baby hamster kidney cells (BHK), human embryonic kidney cells (HEK), PERC.6 (human retinal cells), HBEC30KT cells, and HBEC30KT-shTP53; or plant cells may be used.

In order to induce base conversion in the cell library, base editors may be introduced. The base editors may be introduced into cells by a vector, or the base editors may be introduced into the cell by itself, and the method of introduction is not limited as long as the base editors may show activity in the cell. Here, the description of the vector is as described above.

In the cell library, base conversion may occur by base editors and oligonucleotides including the introduced sgRNAs and the target sequence. That is, gene editing may occur with respect to the introduced target sequence.

In the above method, the transduced cells may be harvested at days 10 and 24 after culturing the cells.

The method of obtaining DNA from a cell library may be performed by using various DNA isolation methods known in the art.

Since each cell constituting the cell library is expected to have genes edited in the introduced target sequence, the target sequence may be sequenced to detect the base conversion efficiency. The sequence analysis method is not limited to a specific method within the range that base conversion efficiency data may be obtained, but for example, deep sequencing may be used.

“Data on the efficiency of base conversion” may be existing known data, or data directly obtained by any method that may be appropriately adopted by those skilled in the art, and the method by which the data is obtained is not limited as long as the data is capable of generating a predictive model that can predict the base conversion efficiency. In an embodiment, the data may be data on the efficiency of base conversion analyzed by using guide RNAs and target sequences corresponding thereto through high-throughput experiments. The efficiency of the base conversion may be derived by Equation 4 below.

$$Base conversion efficiency ( \% ) = \frac{\begin{matrix}Reads of intended \\ ( A > G or C > T ) base conversion\end{matrix}}{Total reads in sorting barcode} . \qquad ( Equation 4 )$$

Analysis of the measured data in the method may include classifying data as valid when the efficiency of base conversion and the frequency of protein mutations due to the base conversion meet criteria, and analyzing the same.

The frequency of protein mutation due to the base conversion may refer to the frequency of protein mutation that appears due to a single nucleotide variant (SNV) caused by conversion of a single nucleotide.

The criteria may be that the efficiency of base conversion in the target sequence is 60% or more; and the frequency of the intended protein mutation is 75% or more compared to the frequency of the unintended protein mutation. In an embodiment, when the efficiency of base conversion in the target sequence by using a base editor is 60%, that is, when base conversion occurs in 60% or more of the total reads, and the intended primary protein mutation due to the same is 75% or more of the total mutations, the efficiency of the high-throughput method using sgRNA may be determined to be the same as the efficiency of the actual single amino acid mutation, and the effects on the cells may be effectively classified into growth (proliferation or outgrowing, interchangeable herein) or depleting through this.

In the present specification, variation and mutation may be used interchangeably.

As used herein, “outgrowing” refers to a state in which cells can proliferate, and for example, may mean a mutation that induces proliferation of cancer cells, which means the number of the cells increase due to the mutation compared to the wild-type.

“Neutral” may mean a mutation that does not affect cell proliferation. In addition, “depleting” is a state in which cells do not proliferate, and the term may be used to mean the opposite of cell proliferation, and refers to a state in which cells do not grow, and in the present specification, the term may be used as meaning the opposite of “outgrowing” mutation.

In an embodiment, for example, when the frequency of base-edited mutations decreases and the frequency of the wild-type in which base-editing does not appear increases, the mutation may be classified as a “outgrowing” mutation, conversely, when the frequency of base-edited mutations increases and the frequency of the wild-type without a base-editing decreases, the mutation may be classified as a “depleting” mutation.

In the method described herein, analysis of sequences and measurement and reading of sequence reads may be performed by using, for example, a next-generation sequencing (NGS) method to obtain UMI counts. Specifically, obtaining an UMI count in the method may be performed by reading UMIs randomly placed in a corresponding position in the numerous reads measured by NGS and counting the number of the UMIs.

In this method, in order to increase the accuracy of UMI counts, 8-nt UMI sequences may be calculated and analyzed according to the alignment barcode using in-house Python scripts. In addition, the UMI count may be normalized and used, and may be interpreted through MAGECK (MAGeCK 0.5.9.3) analysis. In addition, in the disclosure, in order to improve classification accuracy, data having a UMI count of less than 50 at day 10 after culturing the transduced cells may be excluded from the analysis.

In the method, evaluating the function of the mutation may include classifying each mutation as an outgrowing or depleting(depletion) mutation. In addition, the process of evaluating the function of the mutation, as confirmed in an embodiment, may evaluate the anticancer drug resistance-related mutation, and thus, may include the process of classifying the mutation conferring resistance to the EGFR inhibitor afatinib.

In addition, the method is capable of classifying functions of mutations other than depleting and outgrowing, which are the values between depleting and outgrowing, and an evaluation method classifying functions of the mutation as likely depleting, likely neutral (possibly depleting), neutral, likely neutral (possibly outgrowing), and likely outgrowing may be used.

In the method, the positive/negative log fold change (LFC) and P-value of sgRNA may be used to evaluate the function of the mutation, which may be obtained by the MAGECK algorithm. In addition, in order to evaluate the function of the mutation in the method, UMI CPM (number per million) LFC obtained by a UMI count number analysis may be additionally used to perform classification into outgrowing and depleting mutations more accurately. Such a UMI CPM log fold change (LFC) may be obtained by Equations 6 and 7 below:

$$CPM = \frac{individual UMI count}{total UMI counts} * {10}^{6} , \qquad ( Equation 6 )$$

$$Log Fold Change = {log}_{2} \frac{{CPM}_{day 24} + 1}{{CPM}_{day 10} + 1} . \qquad ( Equation 7 )$$

Also, the method may be implemented in a system using a computer.

An aspect provides a system for evaluating cancer mutations including: an information input unit for receiving data of the base conversion efficiency by base editors and the frequency level of protein mutation caused by a base conversion; a data classification unit for classifying data as valid data in case the data received from the information input unit meet criteria; and a data evaluation unit that analyzes the data classified by the data classification unit and analyzes the measured data to evaluate the function of the mutation.

In the system, the base editor may be cytosine base editors (CBEs) and adenine base editors (ABEs).

Since the system uses a configuration utilizing the above-described method, descriptions of the common content between the two are omitted in order to avoid excessive complexity of the present specification, but the description of the overlapping configuration is the same as described above.

Furthermore, in the system, the criterion may be that the efficiency of base conversion in the target sequence is 60% or more; and the frequency of the intended protein mutation is 75% or more compared to the frequency of the unintended protein mutation.

In addition, in the system, the data of the base conversion efficiency by base editors and the frequency level of protein mutation through the base conversion may be obtained by: generating a cell library including a nucleotide sequence encoding a guide RNA, a unique molecular identifier (UMI) nucleotide sequence, and an oligonucleotide including a target nucleotide sequence targeted by the guide RNA; transducing the cell library into cells expressing base editors and culturing; and harvesting the transduced cells after culturing, performing deep sequencing, and measuring the data of the base conversion efficiency and the frequency level of protein mutation due to base conversion.

The harvesting after culturing the transduced cells in the process of measuring the data may be performed at day 10 and 24 after culturing the transduced cells, and deep sequencing may be performed.

The oligonucleotide may further include a barcode sequence. Accordingly, the oligonucleotide may include a sequence encoding an sgRNA, a unique molecular identifier (UMI) nucleotide sequence, a barcode sequence, and a target sequence targeted by the sgRNA. The number of barcode sequences may be one, two, or more. The barcode sequence may be appropriately designed by those skilled in the art according to the purpose. For example, the barcode sequence may be such that enables each sgRNA and a corresponding target sequence pair to be identified after deep sequencing is conducted.

The data evaluation unit for evaluating the function of the mutation in the system may classify each mutation as an outgrowing or depleting(depletion) mutation. In addition, the data evaluation unit for evaluating the function of the mutation, as confirmed in an embodiment, may evaluate the anticancer drug resistance-related mutation, and thus, may classify the mutation conferring resistance to the EGFR inhibitor afatinib.

The system may further include an output unit for outputting the data evaluated by the data evaluation unit. Information on the functional evaluation of the mutations output by the output unit may be expressed as a numerical value calculated for the efficiency of base conversion, or a numerical value relative to a preset reference value, but the form or type of output information is not limited.

Another aspect provides a computer-readable recording medium in which a program is recorded for executing the method of evaluating the function of the cancer mutation by a computer.

Since the recording medium uses the above-described method, descriptions of the common content between the two are omitted in order to avoid excessive complexity of the present specification.

The program may implement the cancer mutation evaluation system or the method of evaluating the function of the cancer mutation with a computer programming language.

Computer programming languages capable of implementing the program include Python, C, C++, Java, Fortran, Visual Basic, and the like, but are not limited thereto. The program may be stored in a recording medium such as a USB memory, compact disc read only memory (CDROM), hard disk, magnetic diskette, or similar medium or device, and may be connected to an internal or external network system. For example, the computer system may access sequence databases such as GenBank (http://www.ncbi.nlm.nih.gov/nucleotide) or Catalog of Somatic Mutations in Cancer (COSMIC) using HTTP, HTTPS, or XML protocols to search the nucleic acid sequence of the target gene and the regulatory region of the gene.

The program may be provided online or offline.

Hereinafter, the present disclosure will be described in more detail through experimental examples and embodiments. However, the experimental examples and embodiments are merely given to exemplify the present disclosure, and the scope of the present disclosure is not limited thereto.

### Experimental Example

### 1. Design of Libraries C and A

Single-nucleotide variants (SNVs) found in human cancer tissues were extracted from the catalog of somatic mutations in cancer (COSMIC) database (release version 84). Mutations listed in COSMIC were accessed from the website in March 2018. 458,189 C>T SNVs and 255,580 A>G SNVs found in human cancer were acquired from the database. To achieve a high frequency of base editing, a highly active 4-bp activity window was designed, which spans protospacer positions 4 to 7, numbered such that the end distal to the NGG PAM is designated as position 1 for both CBE and ABE. 153,425 C>T and 35,163 A>G SNVs that may be generated using CBE and ABE, respectively, were identified.

Next, all mutations with BsmBI cut sites in the sgRNA sequences and corresponding genomic target sequences were filtered out. After filtering out synonymous SNVs, SNVs that may not be generated at high efficiency were removed. Given that the base editing efficiency is usually low when the Cas9 nuclease activity is low, the 10% of the target sequences with the lowest DeepSpCas9 scores, which represent computationally predicted SpCas9 activities, were removed. After these processes, 80,203 and 23,008 sgRNAs were primarily selected, which may induce 84,806 C>T SNVs and 23,176 A>G SNVs using CBE and ABE, respectively.

As negative controls, 500 sgRNAs were added into library C and 139 sgRNAs were added into library A. These sgRNAs do not target any sequence in the human genome (non-targeting control sgRNAs) and have been used as negative controls in genome-wide Cas9-induced knockout screening in human cells. Synonymous mutation-inducing sgRNAs were used as another type of negative control, and 3,028 such sgRNAs were included in library C and 466 sgRNAs were included in library A. This group of sgRNAs is able to induce synonymous SNVs found in the Cancer Gene Census of 719 genes, which represents an expertly curated catalogue of genes that have been implicated in cancer evolution.

### 2. Cell Lines and Culture

HBEC3OKT (RRID: CVCL-AS83) cells are normal human bronchial epithelial cells that were immortalized by the stable expression of CDK4 and hTERT. These cells exhibit intact contact inhibition of proliferation and lack tumorigenic potential. HBEC3OKT-shTP53 cells (P cells) were generated by lentiviral delivery of TP53-targeting shRNA into HBEC3OKT cells. Immunoblot analysis of the products of oncogenic genes such as TP53, KRAS, and LKB1 (STK11) showed that P cells resemble their normal matched control HBEC3OKT cells except for reduced expression of the p53 protein.

P cells were cultured in ACL4 medium (RPMI 1640 (GIBCO, 2.05 mM L-glutamine) supplemented with 0.02 mg/ml insulin, 0.01 mg/ml transferrin, 25 nM sodium selenite, 50 nM hydrocortisone, 10 mM HEPES, 1 ng/ml EGF, 0.01 mM ethanolamine, 0.01 mM O-phosphorylethanolamine, 0.1 nM triiodothyronine, 2 mg/ml bovine serum albumin, 0.5 mM sodium pyruvate) with 2% Tet system-approved fetal bovine serum (FBS, Clontech) and 1% Penicillin-Streptomycin (GIBCO) at 37° C. with 5% CO₂. HEK293T cells (American Type Culture Collection) were cultured in Dulbecco's modified Eagle's Medium (DMEM, GIBCO) with 10% FBS (GIBCO) at 37° C. with 5% CO₂.

### 3. Cloning

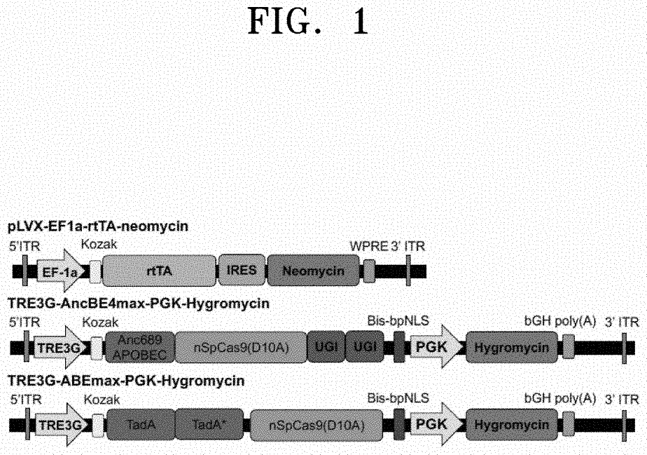

All primers used for cloning are listed in Table 1. All nucleotides were purchased from Macrogen (South Korea). The schematics of each viral vector are shown in FIG. 1.

TABLE 1

| SEQUENCE | | |
|---|---|---|
| ID | | |
| Number | Primer | Sequence |
| 1 | BEnt-erm- | CCACAACACTTTTGTCTTATACTTGGCCGCCACCATGAAAC |
| | FP | GGAC |
| 2 | BEnt-erm- | AGTTCCAGGGGGTGATGGTTTCCTCGCTCTTTCTGGTCAT |
| | RP | CCAGG |
| 3 | BE-Cterm- | ATGACCAGAAAGAGCGAGGAAACCATCACCCCCTGGAACT |
| | FP | TCGAG |
| 4 | BE-Cterm- | ATTCCATATGACGCGTCCCGGGATCTTAGACTTTCCTCTTC |
| | RP | TTCTTGGGCTCG |
| 5 | TRE3G- | GATCCCGGGACGCGTCATATGGAATT |
| | PGK-FP | |
| 6 | TRE3G- | CGCGGTGAGTTCAGGCTTTTTCATGGTAAGCTTGGGCTGC |
| | PGK-RP | AGGTCG |
| 7 | lenti-hygro- | ATGAAAAAGCCTGAACTCAC |
| | FP | |
| 8 | lenti-hygro- | TCATTATTCCTTTGCCCTCGGACGAG |
| | RP | |
| 9 | WPRE-FP | TCCGAGGGCAAAGGAATAATGACGGGGCGCGTCTGGAAC |
| | | AATCA |
| 10 | WPRE-RP | CAACACAGGCGAGCAGCCATGGAAAGGACGTCAGCTTCC |
| 11 | EF1a-FP | GGTAGTCTCAAGCTGGCCGGCCTGCTCTGGTGCCTGGCC |
| | | TCGC |
| 12 | EF1a-RP | GAGTAGTGAGAAATTCGTGGCACCAGATCCTCTAGACTGC |
| | | AGATCGGCACCGGGCTTGCGGGTC |
| 13 | p2A-EGFP- | GCCACGAATTTCTCACTACTCAAGCAGGCCGGTGATGTCG |
| | FP | AGGAAAACCCTGGTCCTGTGAGCAAGGGCGAGGAGCT |
| 14 | p2A-EGFP- | GATTGTCGACTTAACGCGTTTACTTGTACAGCTCGTCCATG |
| | RP | |
| 15 | WPRE- | CATGGACGAGCTGTACAAGTAAACGCGTTAAGTCGACAAT |
| | LTR-FP | CA |
| 16 | WPRE- | AAAAAAATTAGTCAGCCATGGGGCGGAGAATGGGCGGAA |
| | LTR-RP | C |
| 17 | Oligo- | TTGAAAGTATTTCGATTTCTTGGCTTTATATATCTTGTGGAA |
| | Amplifying- | AGGACGAAACACC |
| | FP | |
| 18 | Oligo- | GAGTAAGCTGACCGCTGAAGTACAAGTGGTAGAGTAGAGA |
| | Amplifying- | TCTAGTTACGCCAAGCT |
| | RP | |
| 19 | Improved | GTTTCAGAGCTATGCTGGAAACAGCATAGCAAGTTGAAATA |
| | scaffols | AGGCTAGTCCGTTATCAACTTGAAAAAGTGGCACCGAGTC |
| | with UMI | GGTGCTTTTTTNNNNNNNNTTTGGGAGACGCGATCGAT |
| 20 | Scaffold- | CAAGCTTGGTACCGAGCTCGTTTTCGTCTCTGTTTCAGAG |
| | Amplifying | CTATGCTGG |
| | FP | |
| 21 | Scaffold- | TATAGGGCGAATTGGGCCCTATCGATCGCGTCTCCCAAA |
| | Amplifying- | |
| | RP | |
| 22 | 1st Deep | ACACTCTTTCCCTACACGACGCTCTTCCGATCTCTTGAAAA |
| | sequencing | AGTGGCACCGAGTCG |
| | FP-A | |
| 23 | 1st Deep | ACACTCTTTCCCTACACGACGCTCTTCCGATCTTCTTGAAA |
| | sequencing | AAGTGGCACCGAGTCG |
| | FP-B | |
| 24 | 1st Deep | ACACTCTTTCCCTACACGACGCTCTTCCGATCTCGCTTGAA |
| | sequencing | AAAGTGGCACCGAGTCG |
| | FP-C | |
| 25 | 1st Deep | GTGACTGGAGTTCAGACGTGTGCTCTTCCGATCTTTAAGT |
| | sequencing | CGAGTAAGCTGACCGCTGAAG |
| | RP-A | |
| 26 | 1st Deep | GTGACTGGAGTTCAGACGTGTGCTCTTCCGATCTATTAAGT |
| | sequencing | CGAGTAAGCTGACCGCTGAAG |
| | RP-B | |
| 27 | 1st Deep | GTGACTGGAGTTCAGACGTGTGCTCTTCCGATCTTATTAAG |
| | sequencing | TCGAGTAAGCTGACCGCTGAAG |
| | RP-C | |
| 28 | 2nd | AATGATACGGCGACCACCGAGATCTACAC (8 bp barcode) |
| and | Illuimina | ACACTCTTTCCCTACACGAC |
| 29 | indexing FP | |
| 30 | 2nd | CAAGCAGAAGACGGCATACGAGAT (8 bp barcode) |
| and | Illuimina | GTGACTGGAGTTCAGACGTGT |
| 31 | indexing | |
| | RP | |

To generate pLenti-TRE3G-AncCBE4max-PGK-hygro, the following six DNA fragments were combined using Gibson assembly: (i) BamHI-Ncol digested pLVX-TRE3G (Clontech, 631187)-based lentiviral backbone, (ii) sequences encoding AncAPOBEC1 and the N-terminal region of nCas9 (D10A) amplified via PCR from pCMV-AncBE4max²⁵ (Addgene #112094) using primers of SEQ ID NO :1 and 2, (iii) sequences encoding the C-terminal region of nCas9 (D10A) and 2× uracil glycosylase inhibitor amplified from pCMV-AncBE4max using primers of SEQ ID NO : 3 and 4, (iv) the PGK promoter amplified from pLVX-TRE3G using primers of SEQ ID NO :5 and 6, (v) the hygromycin resistance gene amplified from pLenti HRE Luc pGK Hygro (Addgene #118706) using primers of SEQ ID NO :7 and 8, and (vi) the WPRE element amplified from pLVX-TRE3G using primers of SEQ ID NO :9 and 10.

To generate pLenti-TRE3G-ABEmax-PGK-hygro, the following six DNA fragments were combined using Gibson assembly: (i) BamHI-Ncol digested pLVX-TRE3G (Clontech, 631187)-based lentiviral backbone, (ii) sequences encoding ecTadA and the N-terminal region of nCas9 (D10A) amplified from pCMV-ABE4max²⁵ (Addgene #112098) using primers of SEQ ID NO :1 and 2, (iii) sequences encoding the C-terminal part of nCas9 (D10A) amplified from pCMV-ABE4max using primers of SEQ ID NO :3 and 4, (iv) the PGK promoter amplified from pLVX-TRE3G using primers of SEQ ID NO :5 and 6, (v) the hygromycin resistance gene amplified from pLenti HRE Luc pGK Hygro (Addgene #118706) using primers of SEQ ID NO :7 and 8, and (vi) the WPRE element amplified from pLVX-TRE3G using primers of SEQ ID NO :9 and 10.

To generate pLenti-Guide-Puro-p2A-EGFP, the following four DNA fragments were combined using Gibson assembly: (i) FseI-NcoI digested lentiguide-puro (Addgene, 52963)-based lentiviral backbone, (ii) the sequence encoding EF-1a and the puromycin resistance gene amplified from lentiguide-puro using primers of SEQ ID NO :11 and 12, (iii) sequences encoding p2A and enhanced green fluorescent protein (EGFP) amplified from pCMV-AncBE4max-p2A-GFP (Addgene #112100) using primers of SEQ ID NO :13 and 14, and (iv) WPRE and LTR segments amplified from lentiguide-puro using primers of SEQ ID NO :15 and 16.

All PCR-amplified DNA fragments were generated using a Phusion High-Fidelity DNA polymerase (NEB) with 25 cycles of amplification and an annealing temperature of 60° C., after which they were size-selected by electrophoresis on a 1% agarose gel. The Gibson assembly reaction was performed using NEBuilder HiFi DNA Assembly Master Mix (NEB).

### 4. Lentivirus Production

HEK293T cells were seeded in 100 mm-culture dishes at a density of 5×10⁶ cells per dish 24 hours before transfection. On the day of transfection, the growth medium was exchanged for 10 mL of DMEM containing 25 μM chloroquine diphosphate, after which cells were cultured for 5 hours. Transfer plasmids containing the gene of interest, psPAX2, and pMD2.G were mixed at a molar ratio of 1.64:1.3:0.72 pmol and diluted into 500 μL of Opti-MEM (Life Technology). Polyethylenimine (PEI) was diluted into Opti-MEM in a total volume of 500 μL and added to the DNA mixture such that the ratio of μg DNA:μg PEI was 1:3, resulting in a total volume of 1000 μL. The mixture was incubated for 20 min and added to cells. To achieve a high viral titer, caffeine (Sigma-Aldrich, C0750) was added to the culture medium, at a final concentration of 4 mM, after treatment with the PEI:DNA mixture as previously described. At 12 hours post-transfection, 10 mL of growth medium supplemented with 4 mM of caffeine was added to culture the cells. After 24 hours, the growth medium was harvested and centrifuged at 2,000 g for 10 min to pellet cell debris. The supernatant was filtered through a Millex-HV 0.45-μm low protein-binding membrane (Millipore), divided into aliquots, and kept frozen at −80° C. until use.

### 5. Generation of Cell Lines Expressing Base Editors

1.8 million P cells were plated in 6-well culture plates, with 1.5×10⁵ cells per well. The cells were infected with virus carrying sequences encoding the EF1a-rtTA (reverse tetracycline-controlled transactivator) with the neomycin resistant gene (pLVX-EF1a-Tet3G (Clontech, 631359)) supplemented with 10 μg/ml polybrene (Sigma-Aldrich) at a multiplicity of infection (MOI) of 0.4. The 6-well plates were centrifuged at 1,000 g for 2 hours at 37° C. After centrifugation, the cells were incubated overnight and then refreshed with growth medium containing 1.0 mg/mL of G418 disulfate salt (Sigma-Aldrich). After 10 days from selection, P cells containing rtTA (P-rtTA) were maintained and used as cell lines expressing base editors.

To generate cell lines expressing base editors, 1.8 million P-rtTA cells were plated in 6-well culture plates, with 1.5×10⁵ cells per well. The cells were infected with virus carrying sequences encoding a doxycycline-dependent base editor (Lenti-TRE3G-AncCB E4max-PGK-hygro or Lenti-TRE3G-ABEmax-PGK-hygro) as described above. The day after transduction, cells were refreshed with growth medium containing 80 μg/ml of Hygromycin B Gold (InvivoGen). After 10 days from selection, the obtained base editor-expressing cell lines (P-C or P-A cells) were aliquoted and used for screening.

### 6. Plasmid Library Construction

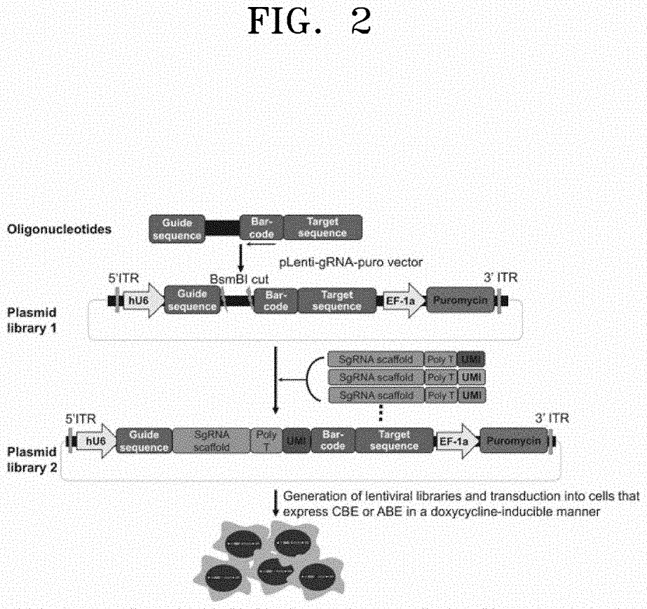

Pooled, 150-nt oligonucleotides for plasmid construction were array-synthesized by Twist Bioscience. Each plasmid in the library was designed to include the following elements, and the process was illustrated in FIG. 2: (i) a 19-nt homology arm with a U6 promoter at the 3′ terminus, (ii) a 20-nt sequence with a G at the 5′ terminus followed by a 19-nt sgRNA guide sequence, (iii) a random 20-nt sequence flanked by a BsmBI cut site on either side (11 nt each), (iv) a 20-nt (libraries C, A, and A1) or 19-nt (libraries C1 and C2) unique barcode sequence corresponding to each sgRNA, (v) a PAM (a 4+23-nt target sequence and a PAM+3-nt), which is identical to an endogenous genomic target locus, and (vi) a 20-nt homology arm.

The pooled oligonucleotides were amplified using primers of SEQ ID NO :17 and 18 and Phusion High-Fidelity DNA Polymerase (NEB), after which they were size-selected by electrophoresis on a 2% agarose gel. The amplicons were assembled into linearized Lenti-gRNA-Puro (Addgene, 84752) after digestion with BsmBI using NEBuilder HiFi DNA Assembly Master Mix (NEB). 200 ng of linearized vector and 120 ng of purified oligonucleotides were used in one Gibson assembly reaction (with a total volume of 20 μL). A total of 16 and 8 reactions were performed for the CBE and ABE libraries (designated the C and A libraries), respectively. After the assembly reactions, the mixtures were pooled and concentrated using a MEGAquick-spin Total Fragment DNA Purification kit (iNtRON Biotechnology, South Korea) and used in up to 12 and 8 electroporation reactions to maximize the library complexity.

An improved form of sgRNA scaffold and a UMI were synthesized (IDT, Primer of SEQ ID NO :19) and amplified using primers of SEQ ID NO :20 and 21. The resulting amplicon was digested with BsmBI and purified. A ligation reaction was then performed using 60 ng of the sticky-ended sgRNA scaffold-UMI fragments and 250 ng of the scaffoldless plasmid library generated above, also digested with BsmBI. A total of 16 and 8 reactions were performed for the CBE and ABE libraries, respectively. The reaction mixtures were pooled, concentrated, and used in up to 12 and 8 electroporation reactions.

### 7. High-Throughput Evaluation of SNVs using CBE and ABE

Twenty-four hours before transduction of lentiviral libraries C and A, 168 million P-C cells and 48 million P-A cells were seeded in duplicate, resulting in 2000-fold coverage of the sgRNA libraries (i.e., on average 2,000 cells/sgRNA) in each replicate. In different replicates, cells were transduced with different batches of the lentiviral library on different days. The cells in each replicate were infected with lentiviral library C or A with 10 μg/ml of polybrene at an MOI of 0.3, such that every sgRNA was represented in approximately 600 cells. 24 hours after the infection, the medium was replaced with fresh medium containing 20 μg/ml of puromycin (Invitrogen) and 2 μg/ml of doxycycline hyclate (Sigma) to induce expression of CBE or ABE; cells were cultured under these conditions for an additional 9 days, and were harvested at day 10 post-infection with approximately 1000-fold to 1500-fold coverage of the sgRNA libraries. The concentration of puromycin in the medium was abnormally high because un-transduced P cells express low levels of the puromycin resistance gene used for immortalization of bronchial epithelial cells. The remaining cells were maintained at numbers sufficient for 2000-fold coverage of the sgRNA libraries (i.e., P-C cells, 83,731 sgRNAs×2,000 cells/sgRNA=˜168 million cells; P-A cells, 23,613 sgRNAs×2,000 cells/sgRNA=˜48 million cells) for an additional 14 days. At day 24 post-infection, the cells were collected for genomic DNA extraction.

In experiments involving small libraries C1, C2, and A1, the same methods were used, except that the cells were maintained at numbers sufficient for 10,000-fold coverage of the sgRNA libraries throughout the experiments. In experiments involving libraries C3, A2, and dA, the same methods were used except that the cells were maintained at numbers sufficient for 3,000-fold coverage of the sgRNA libraries.

In experiments involving library eC, 24 million P-C cells were seeded in duplicate, resulting in 6000-fold coverage of the sgRNA library in each replicate. In different replicates, cells were transduced with different batches of the lentiviral library on different days. The cells were infected with the lentiviral library as described above, after which the medium was replaced with medium containing 20 μg/ml of puromycin (Invitrogen) and 2 μg/ml of doxycycline hyclate (Sigma). The cells were incubated for an additional 9 days, and were harvested at day 10 post-infection with approximately 2,000-fold coverage of the sgRNA library. Upon removal of puromycin and doxycycline, the cell population was split into drug and untreated arms at a representation of 2,000 cells per sgRNA and maintained at numbers sufficient for at least 3,000 fold coverage of the sgRNA library. Cells in the drug arm were cultured and passaged every 3 to 4 days with EGF-free ACL-4 medium containing 10 nM afatinib (Santa Cruz Biotechnology, Dallas, Tex.) for an additional 10 days. Cells in the untreated arm were cultured with complete ACL-4 medium for 10 days.

### 8. Genomic DNA Preparation and Deep Sequencing

Genomic DNA was extracted using a Wizard Genomic DNA Purification Kit (Promega) according to the manufacturer's protocol.

Using the isolated genomic DNA as template, the integrated barcode and target sequences were amplified and prepared for deep sequencing through two PCR steps using 2× Pfu PCR Smart mix (Solgent). In the first step, genomic DNA was divided into multiple 50-μl reactions containing 2.5 μg of genomic DNA, 2050-μl reactions pmol of forward primer mixture of SEQ ID NO :22, 23, and 24, 20 pmol of reverse primer mixture of SEQ ID NO :25, 26, and 27, and 25 μL of PCR premix. The PCR cycling parameters were as follows: an initiation 2 min at 95° C.; followed by 30 s at 95° C., 30 s at 60° C., and 40 s at 72° C., for 24 cycles; and a final 5 min extension at 72 ° C. The total amount of genomic DNA for each experiment represented more than 1000× coverage of the library, assuming 6.6 μg of genomic DNA per 10⁶ cells.

i) Library C: 360 separate 50-μl reactions per replicate experiment (900 μg of DNA, ˜1600× coverage)

ii) Library A: 96 separate 50-μl reactions per replicate experiment (240 μg of DNA, ˜1500× coverage)

iii) Library C1: 40 separate 50-μl reactions per replicate experiment (100 μg of DNA, ˜3800× coverage)

iv) Library A1: 20 separate 50-μl reactions per replicate experiment (50 μg of DNA, ˜3800× coverage)

v) Library C2: 80 separate 50-μl reactions per replicate experiment (200 μg of DNA, ˜7,600× coverage)

vi) Library C3: 80 separate 50-μl reactions per replicate experiment (200 μg of DNA, ˜15,000× coverage)

vii) Library A2: 80 separate 50-μl reactions per replicate experiment (200 μg of DNA, ˜15,000× coverage)

vii) Library eC: 80 separate 50-μl reactions per replicate experiment (200 μg of DNA, ˜7,500× coverage)

ix) Library dA: 80 separate 50-μl reactions per replicate experiment (200 μg of DNA, ˜7,600× coverage)

Amplicons for each experiment were pooled and concentrated with a MEGAquick-spin Total Fragment DNA Purification kit, and size-selected with agarose gel electrophoresis.

In the second PCR step, which was performed to attach sequencing adaptors and barcodes, a total of 250 ng of purified PCR product from the first step was used in eight separate 50-μl reactions for screening libraries and a total of 40 ng of the purified PCR product from the first step was used with 20 pmol of Illumina indexing primers of SEQ ID NO : 28 and 29 in two separate 50-μl reactions for the focused libraries. The PCR cycling parameters were as follows: an initiation 2 min at 95° C. ; followed by 30 s at 95° C., 30 s at 60° C., 40 s at 72° C., for 8 cycles; and a final 5 min extension at 72° C. Amplicons for each experiment were size-selected with agarose gel electrophoresis and sequenced using a HiSeq 2500 System (Illumina) and NextSeq 550 system (Illumina).

### 9. Design of Small Libraries

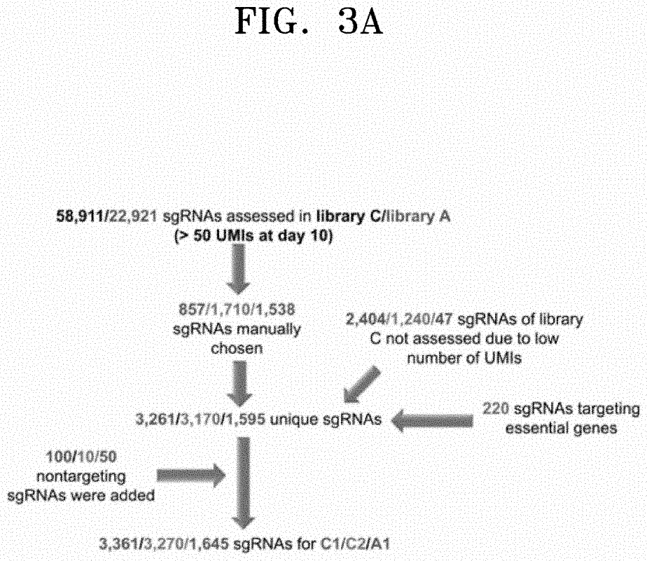

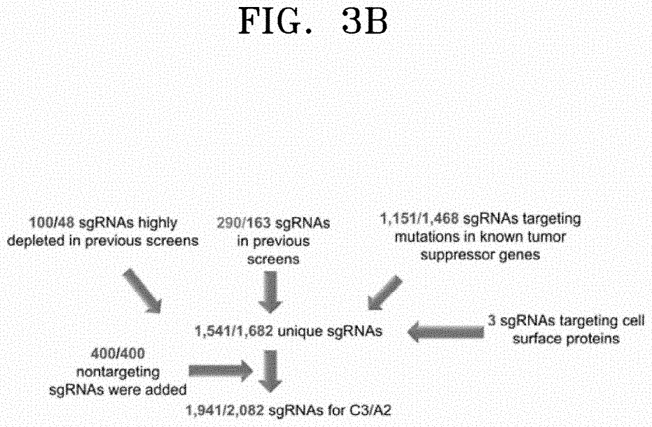

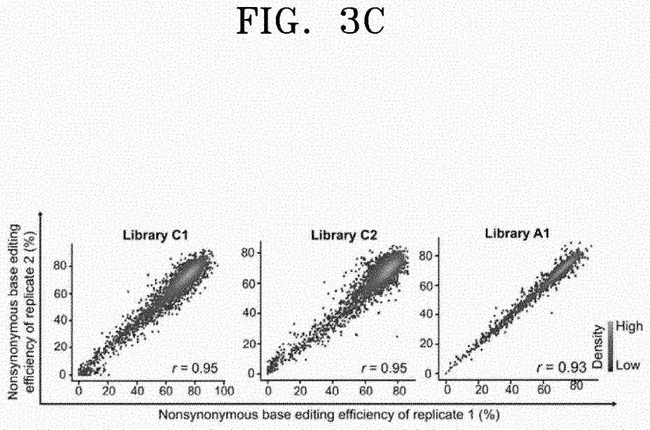

We designed seven independent small libraries named C1 (containing 3,261 sgRNAs), C2 (3,170 sgRNAs), C3 (1,941 sgRNAs), A1 (1,595 sgRNAs), A2 (2,082 sgRNAs), dA (3,136 sgRNAs), and eC (4,157 sgRNAs), and the process of designing the same is shown in FIGS. 3A and 3B.

Libraries C1 and A1: To generate libraries C1 and A1, 857 and 1,538 sgRNAs were randomly chosen from libraries C and A, respectively. Additionally, 2,404 and 47 sgRNAs that were not analyzed due to a low number of UMIs of less than 50 in libraries C and A were included. Finally, 100 and 50 non-targeting sgRNAs were included in each library.

Library C2: To generate library C2, 1,710 sgRNAs were randomly chosen from library C. Additionally, 1,240 sgRNAs that were not analyzed due to a low number of UMIs in library C were included. Also, sgRNAs were included that mediate the disruption of essential genes by generation of a stop codon induced by base editing. As the essential gene candidates, 123 genes included in both a curated set of pan-cancer core fitness genes and the BAGEL essential gene set were selected. From the 124 curated genes, 65 genes 54 related to essential cellular structures and processes were selected: ribosomal proteins (39 genes), DNA replication (2 genes), RNA polymerases (4 genes), proteasomes (8 genes), and spliceosomes (12 genes). sgRNAs that induce stop codons in these genes were designed by using the CRISPR-iSTOP tool, and 220 sgRNAs that were predicted by the DeepCBE tool to induce stop codons with high efficiency were selected. Finally, 100 non-targeting sgRNAs were included in the library.

Libraries C3 and A2: To generate libraries C3 and A2, 100 and 48 sgRNAs that were highly depleted in previous screenings of libraries C/C1/C2 and A/A1, respectively, were initially chosen. Next, 290 and 163 sgRNAs were randomly chosen from previous screenings of libraries C/C1/C2 and A/A1, respectively. Afterwards, sgRNAs designed to induce 1,151 and 1,468 SNVs in known tumor suppressor genes recorded in COSMIC (data release version 84) and the TCGA database (data release version 29.0) were included in libraries C3 and A2, respectively (the SNVs may be generated by a CBE or ABE in the canonical activity window (positions 4 to 8). Finally, 400 non-targeting sgRNAs were included in each library.

Library dA: To generate library dA, 369 high confidence driver genes with high ratios of nonsynonymous to synonymous mutations (dN/dS) were chosen. From these, 2,797 SNVs that cmay be generated by ABE within the canonical activity window (position 4 to 8) were selected. Next, 53 and 23 sgRNAs that had respectively been classified as depleting/likely depleting and outgrowing/likely outgrowing in previous library screenings were included. Finally, 263 non-targeting sgRNAs were included, resulting in 3,136 sgRNAs.

Library eC: 162 genes related to EGF/EGFR signaling pathways were selected. From these, 3,967 SNVs recorded in COSMIC and TCGA that may be generated by CBE within the canonical activity window (position 4 to 8) were chosen, and sgRNAs designed to induce these SNVs were included. Next, 24 sgRNAs with various classifications (6 outgrowing, 4 likely outgrowing, 8 possibly outgrowing, 1 neutral, and 5 not assessed) from previous screenings were included. Finally, 166 non-targeting sgRNAs were included, generating a library including 4,157 sgRNAs.

### 10. Selection of sgRNAs for Individual Functional Evaluations

First, six and seven of the most significantly depleting and outgrowing sgRNAs, that is, six and seven of the sgRNAs with the lowest P-values were selected from the depleting and outgrowing groups, respectively, in the small libraries. All seven of the representative outgrowing sgRNAs were predicted to induce TP53-related mutations (Cg.TP53_p. Q192*, Cg.TP53_p.T155I, Cg.TP53_p. Q100*, Ag.TP53_p.R280G, Ag.TP53_p.N239D, Ag.TP53_p.K120E, Ag.TP53_p.K351E). Five out of the six most significantly depleting sgRNAs were predicted to induce mutations in common essential genes (Cg.POLR1C_p.A6V, Cg.MMS22L_p. R661*, Cg.POLR2B_p.P714L, Ag.CTCF_p.H312R, Ag.SRSF1_p.D139G). Next, four sgRNAs that were annotated as likely outgrowing (Cg.PTPN14_p.Q110* and Cg.CDC23_p.T381M) and likely neutral (Cg.ACOX3_p.Q145* and Cg.KMT2C_p.R1906*) were arbitrarily selected. Additionally, one TP53-related sgRNA (Ag.TP53_p.T125A) that was predicted to introduce a mutation known to destabilize the p53 protein was selected. However, only library C contained the sgRNA and the same was classified as likely outgrowing in library C. Additionally, two sgRNAs classified as likely neutral (possibly outgrowing) were chosen, but the two sgRNAs were predicted to introduce missense mutations in common essential genes (Ag.POLE_p.Y1889C and Ag.ACTL6A_p.T405A), compared to the control group. Finally, eight sgRNAs classified as likely outgrowing or outgrowing were additionally selected. In summary, 28 sgRNAs shown in Table 2 below were chosen for validation.

TABLE 2

| No | Sg.ID | Classification | Target gene |
|---|---|---|---|
| 1 | Cg.TP53_p.Q192* | Likely outgrowing | Tumor suppressor gene |
| 2 | Cg.TP53_p.T1551, p.T155= | Likely outgrowing | Tumor suppressor gene |
| 3 | Cg.TP53_p.Q100*, p.S99F | Likely outgrowing | Tumor suppressor gene |
| 4 | Cg.ACOX3_p.Q145* | Neutral | Enzyme gene |
| 5 | Cg.KMT2C_p.R1906* | Likely neutral | Tumor suppressor gene |
| | | (Possibly outgrowing) | |
| 6 | Cg.CDC23_p.T381M | Likely outgrowing | Cell Cycle gene |
| 7 | Cg.PTPN14_p.Q110* | Likely Outgrowing | Tumor suppressor gene |
| 8 | Cg.POLR1C_p.A6V | Depleting | Common essential gene |
| 9 | Cg.MMS22L_p.R661* | Depleting | Common essential gene |
| 10 | Cg.POLR2B_p.714L | Depleting | Common essential gene |
| 11 | Cg.POLG_p.Q1029* | Likely depleting | DNA polymerase |
| 12 | Ag.TP53_p.R280G | Likely outgrowing | Tumor suppressor gene |
| 13 | Ag.TP53_p.N239D | Outgrowing | Tumor suppressor gene |
| 14 | Ag.TP53_p.K120E, p.K120R | Likely outgrowing | Tumor suppressor gene |
| 15 | Ag.TP53_p.K351E | Likely neutral | Tumor suppressor gene |
| | | (Possibly outgrowing) | |
| 16 | Ag.TP53_p.T125A | Likely outgrowing | Tumor suppressor gene |
| 17 | Ag.CTCF_p.H312R | Depleting | Common essential gene |
| 18 | Ag.SRSF1_p.D139G | Depleting | Common essential gene |
| 19 | Ag.POLE_p.Y1889C | Neutral | Common essential gene |
| 20 | Ag.ACTL6A_p.T405A | Neutral | Common essential gene |
| 21 | Cg.GNA13_p.Q27* | Outgrowing | Oncogene |
| 22 | Cg.CASP8_p.S158F | Outgrowing | Tumor suppressor gene |
| 23 | Cg.PSMB5_p.S261F | Likely neutral | Proteasome subunit |
| | | (Possibly outgrowing) | |
| 24 | Cg.PHLDA1_p.Q201* | Outgrowing | Apoptosis regulation |
| 25 | Ag.PHLDA1_p.Y249C | Likely outgrowing | Apoptosis regulation |
| 26 | Ag.IRF6_Y97C | Outgrowing | Transcription activator |
| 27 | Ag.EGFR_p.Y727C | Outgrowing | Oncogene |
| 28 | Ag.SIK1_p.F26L | Likely outgrowing | Tumor suppressor gene |

sgRNA-encoding sequences were individually cloned into the Lenti-Guide-Puro vector (Addgene, #52963). Cells expressing 1.2 million base editors per sgRNA were seeded in 100-mm culture dishes 24 hours before transduction. The cells were infected in duplicate with lentivirus harboring sequences encoding individual sgRNAs at a low MOI (˜0.4). In addition, cells expressing base editors were seeded as above for a GFP positive control. In this case, lentivirus harboring an empty sgRNA cassette and the puromycin resistance gene-p2A-GFP fusion gene was used to infect cells at a low MOI (˜0.4). The day after transduction, the medium was replaced with fresh medium containing 20 μg/ml of puromycin (Invitrogen) and 2 μg/ml of doxycycline hyclate (Sigma) to induce expression of the base editor; these conditions were maintained for 48 hours. After removal of puromycin, the cells were maintained for an additional 7 days with doxycycline treatment.

### 11. Competitive Proliferation Assay

Ten days after infection, cells transduced with lentivirus encoding candidate hit sgRNAs (GFP−) and cells transduced with the positive control lentivirus (GFP+) were mixed and grown together in triplicate. The cells were sampled every 3 or 4 days and the ratio of GFP+ to GFP− cells in the mixture was quantified via longitudinal flow cytometry. By assuming that the cells exhibit an exponential growth rate, the number of cells (N) at times t1 and t2 may be described by the following Equation 1, where f₀ is the absolute fitness of the reference cells and Δf__{gRNA} is the fitness change caused by the transduced sgRNA:

Nₜ₂=Nₜ₁×2^{(f}^{₀}^{+Δf}^{_{gRNA}}^{)(t}^{₂}^{-t}⁾, (Equation 1).

The ΔfgRNA, between a certain time point tᵢ and the reference time point t₀ was obtained according to the Equation 2:

$$\frac{{N}_{gRNA , ti}}{{N}_{c , ti}} = \frac{{N}_{gRNA , t 0} \times {2}^{( {f}_{o} + Δ {f}_{gRNA , ti} ) ( {t}_{i} - {t}_{o} )}}{{N}_{c , t 0} \times {2}^{( {f}_{o} ) ( {t}_{i} - {t}_{o} )}} . \\ \frac{\frac{{N}_{gRNA , ti}}{{N}_{c , ti}}}{\frac{{N}_{gRNA , t 0}}{{N}_{c , t 0}}} = {2}^{Δ {f}_{gRNA , ti}} . \qquad ( Equation 2 )$$

The ratio between the number of GFP− cells (N_{gRNA}) and the number of GFP+ cells (N_{c}) was obtained from the competitive growth assay, and the relative fitness of the GFP+ cells were assumed to be equal to the fitness of the reference cells (f₀). The relative enrichment (E_{gRNA,ti}) between a certain time point tᵢ and the reference time point t₀ was determined by the following equation:

$${E}_{gRNA , ti} = \frac{\frac{{N}_{gRNA , ti}}{{N}_{c , ti}}}{\frac{{N}_{gRNA , t 0}}{{N}_{c , t 0}}} \times 100 ( \% ) . \qquad ( Equation 3 )$$

### 12. Allele Frequency Tracking After Transduction of an Individual sgRNA

The cells harboring an individual sgRNA and a base editor were seeded in duplicate after removal of doxycycline at 10 days post-infection. These cells were cultured for an additional 2 weeks, and harvested at 10, 17, and 24 days post-infection. Each sgRNA-targeted genomic site was amplified using site-specific primers and analyzed by deep sequencing.

The amplicons for deep sequencing were prepared through three successive rounds of PCR. The first PCR step was performed using 1 μg of genomic DNA, Q5 DNA polymerase (NEB), and 20 pmol of ‘amplifying’ primer in a single 20 μl-reaction. The second step was performed using 3 μL of the first-step PCR product and 20 pmol of the ‘adaptor’ primer in Table 1 above. The primers used in the individual sgRNA experiment are shown in Table 3 below.

TABLE 3

| | GN19 | | | | |
|---|---|---|---|---|---|
| | guide | Amplifying_ | Amplifying_ | | |
| Sg. ID | sequence | FP | RP | Adaptor_FP | Adaptor_RP |
| Cg.TP53_ | GGCA | | | ACACTCTTTCCCT | GTGACTGGAGTTC |
| p.T155l, | ccCG | | | ACACGACGCTCTT | AGACGTGTGCTCT |
| p.T155T | CGTC | | | CCGATCTTTGCCC | TCCGATCTTCTCC |
| | CGC | | | AGGGTCCCCAGG | AGCCCCAGCTGCT |
| | GCCA | | | CC | C |
| | (SEQ | | | (SEQ ID NO: 33) | (SEQ ID NO: 34) |
| | ID | | | | |
| | NO: | | | | |
| | 32) | | | | |
| Cg.TP53_ | CTTc | | | ACACTCTTTCCCT | GTGACTGGAGTTC |
| p.Q100* | CcAG | | | ACACGACGCTCTT | AGACGTGTGCTCT |
| | AAAA | | | CCGATCTCCCCT | TCCGATCTAATGC |
| | CCTA | | | GTCATCTTCTGTC | AGGGGGATACGG |
| | CCA | | | CC | CCA |
| | (SEQ | | | (SEQ ID NO: 36) | (SEQ ID NO: 37) |
| | ID | | | | |
| | NO: | | | | |
| | 35) | | | | |
| Cg.CDC23_ | Aacac | TGGGT | CAAGTG | ACACTCTTTCCCT | GTGACTGGAGTTC |
| p.T381M | gtctgct | GTTTT | ACCAGG | ACACGACGCTCTT | AGACGTGTGCTCT |
| | gctatc | GCCAG | CTTACC | CCGATCTGGTGC | TCCGATCTCTGCC |
| | c | GACTT | GA | CTGGACACTAATG | CGAGGCCATACCA |
| | (SEQ | (SEQ ID | (SEQ ID | GG | AG |
| | ID | NO: 39) | NO: 40) | (SEQ ID NO: 41) | (SEQ ID NO: 42) |
| | NO: | | | | |
| | 38) | | | | |
| Cg.PTPN14_ | GCTT | CTTAC | TTATATG | ACACTCTTTCCCT | GTGACTGGAGTTC |
| p.Q110* | cAGC | CTCAC | GCACAC | ACACGACGCTCTT | AGACGTGTGCTCT |
| | AAGA | ATGGG | AGGGGG | CCGATCTCAAGA | TCCGATCTGGCAC |
| | GGC | CGCTT | A | GCCAGCAAGCAC | ACAGGGGGAAAAT |
| | CACA | (SEQ ID | (SEQ ID | GAT | GC |
| | (SEQ | NO: 44) | NO: 45) | (SEQ ID NO: 46) | (SEQ ID NO: 47) |
| | ID | | | | |
| | NO: | | | | |
| | 43) | | | | |
| Cg.TP53_ | CCTc | | | ACACTCTTTCCCT | GTGACTGGAGTTC |
| p.Q192* | AGCA | | | ACACGACGCTCTT | AGACGTGTGCTCT |
| | TCTT | | | CCGATCTTTGCCC | TCCGATCTAGGGC |
| | ATCC | | | AGGGTCCCCAGG | CACTGACAACCAC |
| | GAG | | | CC | CC |
| | (SEQ | | | (SEQ ID NO: 49) | (SEQ ID NO: 50) |
| | ID | | | | |
| | NO: | | | | |
| | 48) | | | | |
| Cg.POLR1C_ | TCAG | TGGGA | CCCAGG | ACACTCTTTCCCT | GTGACTGGAGTTC |
| p.A6V | GcGG | TCGGC | CATCAT | ACACGACGCTCTT | AGACGTGTGCTCT |
| | TGGA | CGGAA | CATAAC | CCGATCTCTCGC | TCCGATCTACGAA |
| | GGAA | CAC | CGG | GATATTTAAGATT | TTTGTCCACGAAG |
| | ATG | (SEQ ID | (SEQ ID | CCAGGAGGC | GGACAG |
| | (SEQ | NO: 52) | NO: 53) | (SEQ ID NO: 54) | (SEQ ID NO: 55) |
| | ID | | | | |
| | NO: | | | | |
| | 51) | | | | |
| Cg.MMS22L_ | ATGT | GAGAG | CGGATA | ACACTCTTTCCCT | GTGACTGGAGTTC |
| p.R661* | cGAG | CCCTC | TATTCAA | ACACGACGCTCTT | AGACGTGTGCTCT |
| | AATC | ATTTG | CCTGTA | CCGATCTGGTAC | TCCGATCTCTGAA |
| | TGAA | GAAGG | CTGATTT | AGAGACAGACTAT | AACCATACCTGAT |
| | CTT | GTC | ATGCCC | CTGGACCC | TCTGGCC |
| | (SEQ | (SEQ ID | (SEQ ID | (SEQ ID NO: 59) | (SEQ ID NO: 60) |
| | ID | NO: 57) | NO: 58) | | |
| | NO: | | | | |
| | 56) | | | | |
| Cg.POLR2B_ | TTCc | TTAGTT | CATACC | ACACTCTTTCCCT | GTGACTGGAGTTC |
| p.P714L | TGAT | GGCAG | TGCTGG | ACACGACGCTCTT | AGACGTGTGCTCT |
| | CATA | GATCT | CAGCTC | CCGATCTGCAGG | TCCGATCTCCCTC |
| | ACCA | TGTGG | TCTA | ATCTTGTGGCCA | CAGATAAGACTAC |
| | GGT | C | (SEQ ID | GTG | AGAGTTACTTTC |
| | (SEQ | (SEQ ID | NO: 63) | (SEQ ID NO: 64) | (SEQ ID NO: 65) |
| | ID | NO: 62) | | | |
| | NO: | | | | |
| | 61) | | | | |
| Cg.POLG_ | GGTC | TGATAT | TGCTCC | ACACTCTTTCCCT | GTGACTGGAGTTC |
| p.Q1029* | cAGA | GTGAA | AAAGGT | ACACGACGCTCTT | AGACGTGTGCTCT |
| | GAGA | CATTC | AGCAAG | CCGATCTCCCCA | TCCGATCTAGCAT |
| | AACT | CTTGC | ATACCT | GGTATCGGCTGT | CCAAGCTCTTCTG |
| | GCA | CAAGG | C | CG | GGG |
| | (SEQ | C | (SEQ ID | (SEQ ID NO: 69) | (SEQ ID NO: 70) |
| | ID | (SEQ ID | NO: 68) | | |
| | NO: | NO: 67) | | | |
| | 66) | | | | |
| Cg.ACOX3_ | ATTc | ACTCTT | CAAGAA | ACACTCTTTCCCT | GTGACTGGAGTTC |
| p.Q145* | AAAA | CTTAC | GAGCAT | ACACGACGCTCTT | AGACGTGTGCTCT |
| | GATC | CTGCC | AAGCCC | CCGATCTTTCTTA | TCCGATCTAGAAG |
| | TTCA | CCCT | CCT | CCTGCCCCCTGT | AGCATAAGCCCCC |
| | GGA | (SEQ ID | (SEQ ID | TG | TGG |
| | (SEQ | NO: 72) | NO: 73) | (SEQ ID NO: 74) | (SEQ ID NO: 75) |
| | ID | | | | |
| | NO: | | | | |
| | 71) | | | | |
| Cg.KMT2C_ | CCCC | AGACT | CAGGGG | ACACTCTTTCCCT | GTGACTGGAGTTC |
| p.R1906* | TcGA | TCTCA | ATGGCC | ACACGACGCTCTT | AGACGTGTGCTCT |
| | CCAC | GCCAC | TATTTGC | CCGATCTGACTTC | TCCGATCTTCTCC |
| | CTCC | CCTCA | T | TCAGCCACCCTC | AGCCCCAGCTGCT |
| | TGT | (SEQ ID | (SEQ ID | AC | C |
| | (SEQ | NO: 77) | NO: 78) | (SEQ ID NO: 79) | (SEQ ID NO: 80) |
| | ID | | | | |
| | NO: | | | | |
| | 76) | | | | |
| Ag.TP53_ | GGG | | | ACACTCTTTCCCT | GTGACTGGAGTTC |
| p.R280G | AGAG | | | ACACGACGCTCTT | AGACGTGTGCTCT |
| | ACCG | | | CCGATCTGGGAC | TCCGATCTACCGC |
| | GCG | | | AGGTAGGACCTG | TTCTTGTCCTGCTT |
| | CACA | | | ATT | G |
| | G | | | (SEQ ID NO: 82) | (SEQ ID NO: 83) |
| | (SEQ | | | | |
| | ID | | | | |
| | NO: | | | | |
| | 81) | | | | |
| Ag.TP53_ | TGTG | GAAGC | GTAAGG | ACACTCTTTCCCT | GTGACTGGAGTTC |
| p.N239D | TaAC | TTACA | AGATTC | ACACGACGCTCTT | AGACGTGTGCTCT |
| | AGTT | GAGGC | CCCGCC | CCGATCTCTGCTT | TCCGATCTCAGTG |
| | CCTG | TAAGG | GG | GCCACAGGTCTC | TGCAGGGTGGCAA |
| | CAT | GC | (SEQ ID | C | G |
| | (SEQ | (SEQ ID | NO: 86) | (SEQ ID NO: 87) | (SEQ ID NO: 88) |
| | ID | NO: 85) | | | |
| | NO: | | | | |
| | 84) | | | | |
| Ag.TP53_ | GCCA | | | ACACTCTTTCCCT | GTGACTGGAGTTC |
| p.K120E | AGTC | | | ACACGACGCTCTT | AGACGTGTGCTCT |
| | TGTG | | | CCGATCTCCCCT | TCCGATCTAATGC |
| | ACTT | | | GTCATCTTCTGTC | AGGGGGATACGG |
| | GCA | | | CC | CCA |
| | (SEQ | | | (SEQ ID NO: 90) | (SEQ ID NO: 91) |
| | ID | | | | |
| | NO: | | | | |
| | 89) | | | | |
| Ag.TP53_ | ACTC | TGCAT | CTGGGA | ACACTCTTTCCCT | GTGACTGGAGTTC |
| p.K351E | AAGG | GTTGC | CCCAAT | ACACGACGCTCTT | AGACGTGTGCTCT |
| | ATGC | TTTTGT | GAGATG | CCGATCTCTTCTC | TCCGATCTGAAGG |
| | CCAG | ACCGT | GG | CCCCTCCTCTGTT | CAGGGGAGTAGG |
| | GCT | (SEQ ID | (SEQ ID | G | GCC |
| | (SEQ | NO: 93) | NO: 94) | (SEQ ID NO: 95) | (SEQ ID NO: 96) |
| | ID | | | | |
| | NO: | | | | |
| | 92) | | | | |
| Ag.TP53_ | TGCA | | | ACACTCTTTCCCT | GTGACTGGAGTTC |
| p.T125A | CGGT | | | ACACGACGCTCTT | AGACGTGTGCTCT |
| | CAGT | | | CCGATCTCCCCT | TCCGATCTAATGC |
| | TGCC | | | GTCATCTTCTGTC | AGGGGGATACGG |
| | CTG | | | CC | CCA |
| | (SEQ | | | (SEQ ID NO: 98) | (SEQ ID NO: 99) |
| | ID | | | | |
| | NO: | | | | |
| | 97) | | | | |
| Ag.CTCF_ | ATCa | CAGTT | CCAGGC | ACACTCTTTCCCT | GTGACTGGAGTTC |
| p.H312R | CCTT | ACACG | ATCTATT | ACACGACGCTCTT | AGACGTGTGCTCT |
| | AACA | TGTCC | GCCTGA | CCGATCTCAGTTA | TCCGATCTCTTCC |
| | CACA | ACGGC | GAC | CACGTGTCCACG | TTTAAATTCCCGCT |
| | CAC | (SEQ ID | (SEQ ID | GC | GGAGTC |
| | (SEQ | NO: | NO: 102) | (SEQ ID NO: 103) | (SEQ ID NO: 104) |
| | ID | 101) | | | |
| | NO: | | | | |
| | 100) | | | | |
| Ag.SRSF1_ | AGGa | CGAGG | CAACCT | ACACTCTTTCCCT | GTGACTGGAGTTC |
| p.D139G | TCAC | ATTGC | TGCCTG | ACACGACGCTCTT | AGACGTGTGCTCT |
| | ATGC | TGCTG | AATCCTT | CCGATCTCCAGC | TCCGATCTCGTAC |
| | GTGA | TGGTG | ACCTTG | TCTCTTTACCTGG | AAACTCCACGACA |
| | AGC | (SEQ ID | (SEQ ID | TATCACTTAAG | CCAG |
| | (SEQ | NO: | NO: 107) | (SEQ ID NO: 108) | (SEQ ID NO: 109) |
| | ID | 106) | | | |
| | NO: | | | | |
| | 105) | | | | |
| Ag.POLE_ | AGTa | GGACC | CTTCCT | ACACTCTTTCCCT | GTGACTGGAGTTC |
| p.Y1889C | CATC | CTCAG | GAACTT | ACACGACGCTCTT | AGACGTGTGCTCT |
| | ACCA | CTCTTT | GCCCAA | CCGATCTTGGGT | TCCGATCTGCACC |
| | GCAG | TCCC | CTCAAG | GCCCTCTGGCTC | TCAGGGGGTCATT |
| | GTG | (SEQ ID | (SEQ ID | TC | TTAGC |
| | (SEQ | NO: | NO: 112) | (SEQ ID NO: 113) | (SEQ ID NO: 114) |
| | ID | 111) | | | |
| | NO: | | | | |
| | 110) | | | | |
| Ag.ACTL6A_ | GGGT | AGGTG | AGCCTA | ACACTCTTTCCCT | GTGACTGGAGTTC |
| p.T405A | aCCT | GGAGC | AGGTAA | ACACGACGCTCTT | AGACGTGTGCTCT |
| | TTCA | ATCCC | AAAGCA | CCGATCTTGGCT | TCCGATCTGGAAG |
| | ACAG | TTGAA | TAGGCA | GACAGAGCAAGA | GTAGAAGCTTGGG |
| | ATG | C | G | CCTTCTC | AACTC |
| | (SEQ | (SEQ ID | (SEQ ID | (SEQ ID NO: 118) | (SEQ ID NO: 119) |
| | ID | NO: | NO: 117) | | |
| | NO: | 116) | | | |
| | 115) | | | | |

The amplifying primers used in Table 3 were used only when the amplification was poor with the adaptor primers and additional amplification was required. The third step was performed to attach sequencing adaptors and a barcode sequence, using 2 μL of the first-step PCR product and 20 pmol of Illumina indexing primers of SEQ ID NO : 29 and 29. In all cases, the PCR cycling parameters were as follows: an initiation 2 min at 98° C.; followed by 30 s at 98° C., 30 s at 58° C., 1 min 30 s at 72° C., for 20 cycles; and a final 5 min extension at 72° C.

### 13. Analysis of Base Editing Outcomes in Surrogate Target Sequences

Deep sequencing data generated from the library were analyzed by using custom Python scripts, which were used in a previous study (Song, M. et al. Sequence-specific prediction of the efficiencies of adenine and cytosine base editors. Nature biotechnology 38, 1037-1043 (2020)). Guide RNAs and corresponding surrogate sequences were extracted using the ‘Sorting barcode’, including the TTTG sequence (a common 4-nt sequence for the BsmBI restriction site), the unique barcode sequences located upstream of the target sequences (20-nt in length for libraries C, A, and A1; 19-nt in length for libraries C1 and C2), and the 4-nt sequence downstream of the surrogate target sequence (only for libraries C and A). Insertions or deletions located near the 8-nt region surrounding the expected cleavage site were considered to be indels.

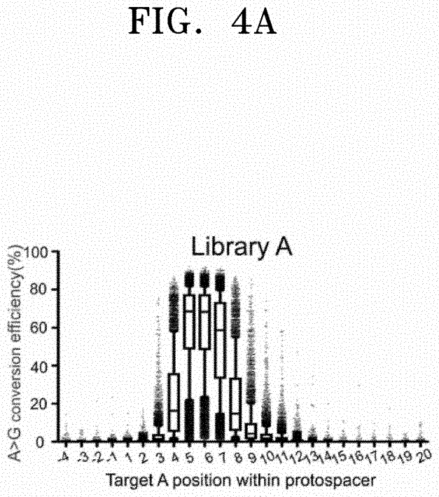

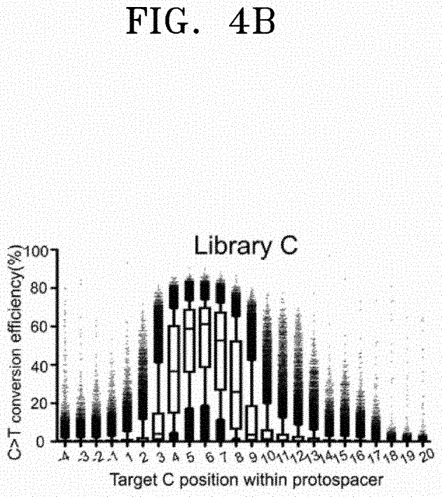

For analysis of base editing efficiencies and allele frequencies, the reads were sorted by their unique barcode sequences and reads containing indels were excluded from further analysis. For ABE and CBE, only base editing of any A converted to G or any C converted to T, respectively, were considered. Any pairs with fewer than 100 reads in both replicates were filtered out, and the A>G or C>T conversion efficiency at each position of each sgRNA target site was calculated with the following equation and the results of identifying the same are shown in FIG. 4.

$$Base conversion efficiency ( \% ) = \frac{\begin{matrix}Reads of intended \\ ( A > G or C > T ) base conversion\end{matrix}}{Total reads in sorting barcode} . \qquad ( Equation 4 )$$

For analysis of the proportion of bases that underwent editing, each barcode-sorted read was analyzed according to the sequence outcomes in the base editing window. We analyzed the full length of the sgRNA target site, from position 1 to 20, to exclude any possibility of unintended amino acid changes outside of the canonical base editing window (spanning positions 4 to 8). The proportion of base editing outcomes was calculated with Equation 5 below:

$$Base editing outcome proportion = \frac{Reads of a specific base edited outcome}{Total reads in the sorting barcode} . \qquad ( Equation 5 )$$

Next, the outcome proportion derived from nucleotide editing was transformed to a codon-based outcome proportion by using a Python script. Nonsynonymous base editing efficiencies were calculated as the sum of any base editing outcomes that changed amino acid codons in the target gene.

### 14. MAGECK Analysis

For UMI analysis, 8-nt UMI sequences were counted and analyzed according to the sorting barcode by using in-house Python scripts. The in-house Python scripts used a directional network to integrate UMIs in order to minimize incorrect identification of UMIs as a result of sequencing errors, referring to the classification criteria presented in a previous study (Clement, K. et al. CRISPResso2 provides accurate and rapid genome editing sequence analysis. Nature biotechnology 37, 224-226 (2019)). Different UMIs were combined when they varied by only one nucleotide and when their read count fold difference was three or more. When the read count fold difference of UMIs that differed by one nucleotide was less than three, UMIs were not combined and were regarded as unique. For UMI-based MAGECK analysis, a UMI read count table was generated that contained the read count of every UMI of each sgRNA. The UMI read count was normalized as RPM. To calculate the fold change and the statistical significance of read count changes between samples of day 24 and day 10, MAGECK (MAGeCK 0.5.9.3) analysis was performed. For such analysis, sgRNAs containing fewer than 50 UMIs at day 10 were excluded to increase the accuracy of functional classification.

Four internal groups of UMI-derived clones (replicate^{UMI}) were used for each sgRNA to calculate the fold changes and significance of the sgRNA on the basis of four internal replicates as described in a previous study (Zhu, S. et al. Guide RNAs with embedded barcodes boost CRISPR-pooled screens. Genome biology 20, 20 (2019)). UMIs were randomly annotated to each replicate^{UMI} so that four replicates^{UMI} had the same or a similar number of UMIs. Then, the median RPM of the samples of day 10 and day 24 in each replicate^{UMI} was calculated and used as input for MAGECK analysis to derive a positive/negative P-value and LFC of an sgRNA. The LFC values calculated from the MAGECK algorithm were median subtracted to obtain a nLFC. Plotting the nLFC (x-axis) versus the negative logarithm of the robust rank aggregation (RRA) P-value (y-axis) produced a volcano plot. The lower value between the negative and positive P-values was selected as the P-value used in the volcano plot. When results from replicates were combined, the percentile rank of the nLFC and P-value were averaged across replicates.

### 15. UMI Count Analysis

UMI count analysis was performed using the fold change of CPM, which was scaled by the total UMI counts between day 10 and day 24. To calculate the log fold change (LFC), a pseudo-count of 1 was added to all counts to handle UMI counts of zero in the samples of day 10 or day 24.

$$CPM = \frac{individual UMI count}{total UMI counts} * {10}^{6} , \qquad ( Equation 6 )$$

$$Log Fold Change = {log}_{2} \frac{{CPM}_{day 24} + 1}{{CPM}_{day 10} + 1} . \qquad ( Equation 7 )$$

### 16. Functional Classification of sgRNAs

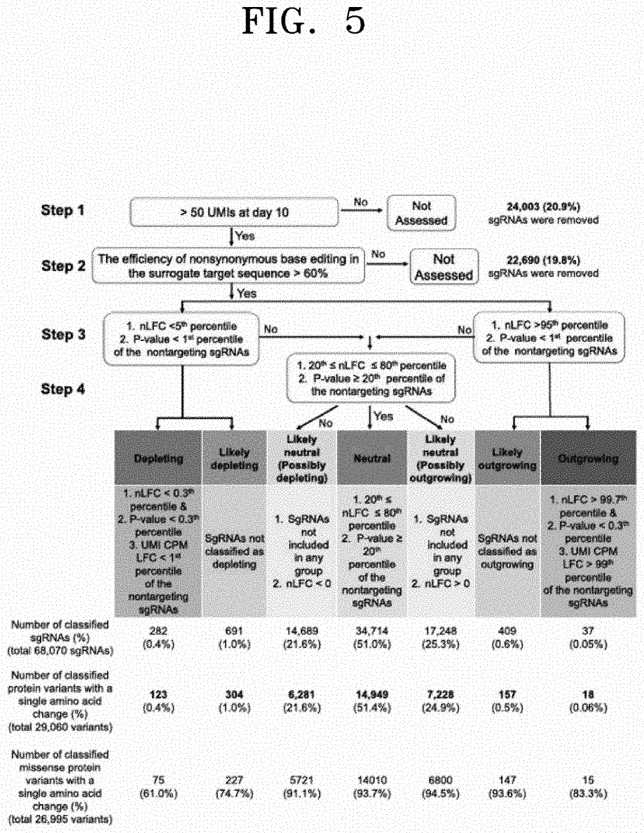

For UMI-based analysis, all UMIs with fewer than 5 raw read counts at day 10 were excluded from further analysis. The functional classification system is summarized in a flowchart in FIG. 5.

As shown in FIG. 5, sgRNAs with 50 or fewer UMIs at day 10 were excluded from further analysis (Step 1). When the nonsynonymous base editing efficiency induced by sgRNA was lower than 60% in the surrogate target sequence, the sgRNAs were also excluded from further analysis (Step 2). The remaining sgRNAs were classified depending on their nLFCs and P-values using cutoff values determined by the distribution of the non-targeting control sgRNAs in each library.

Finally, we classified sgRNAs into 7 groups as follows:

(1) Depleting: sgRNAs whose nLFC and P-value were less than the corresponding values in the 0.3th percentile of the non-targeting sgRNAs and whose UMI CPM fold change was less than the corresponding values in the 1st percentile of the non-targeting sgRNAs.

(2) Likely depleting: sgRNAs whose nLFC and P-value were less than the corresponding values in the 5^{th} and 1^{st} percentiles, respectively, of the non-targeting sgRNAs but that were not classified as depleting.

(3) Likely neutral (Possibly depleting): sgRNAs whose nLFC was less than 0 and that were not classified as depleting, likely depleting, or neutral.

(4) Neutral: sgRNAs whose nLFC was between the corresponding values in the 20^{th} and 80^{th} percentiles and whose P-value was greater than the corresponding values in the 20^{th} percentile of the non-targeting sgRNAs.

(5) Likely neutral (Possibly outgrowing): sgRNAs whose nLFC was greater than 0 and that were not classified as outgrowing, likely outgrowing, or neutral.

(6) Likely outgrowing: sgRNAs whose nLFC was greater than the corresponding values in the 95^{th} percentile and whose P-value was less than the corresponding values in the 1^{st} percentile of the non-targeting sgRNAs but that were not classified as outgrowing.

(7) Outgrowing: sgRNAs whose nLFC was greater than the corresponding values in the 99.7th percentile and whose P-value was less than the corresponding values in the 0.3th percentile of the non-targeting sgRNAs and whose UMI CPM fold change was greater than the corresponding values in the 99^{th} percentile of the non-targeting sgRNAs.

For sgRNAs with two barcodes, UMIs from the two barcodes were combined for subsequent analyses. When the functional classification of a sgRNA differed depending on which sgRNA library was used, the classification from the library that had a higher number of UMIs (UMI CPM) for the sgRNA was chosen. When the relative frequency of the variant allele among the base-edited nonsynonymous sequences in surrogate sequences was higher than 75%, the functional classification of the allele was considered to be the same as that of the corresponding sgRNA.

### 17. Statistical Significance

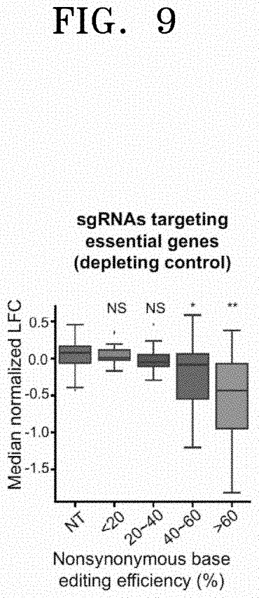

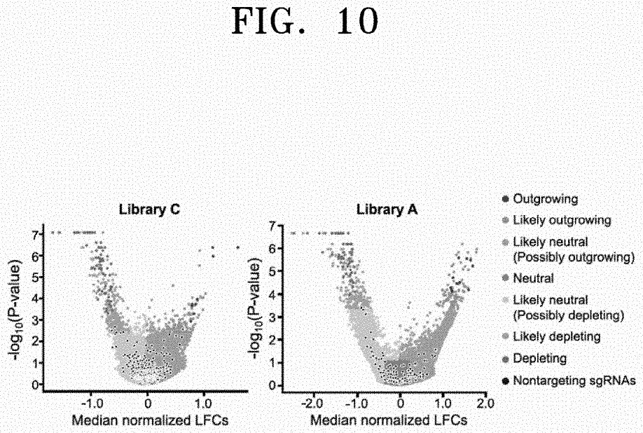

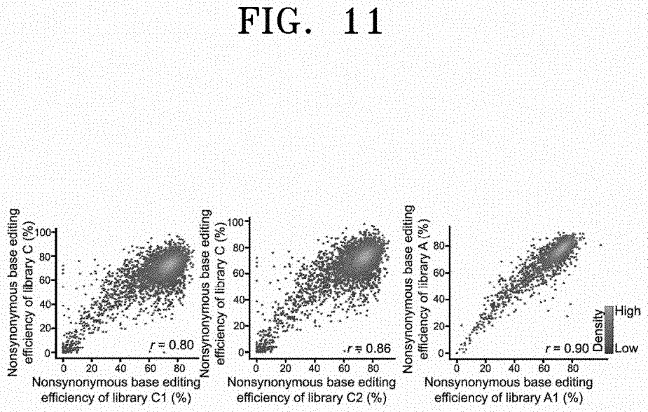

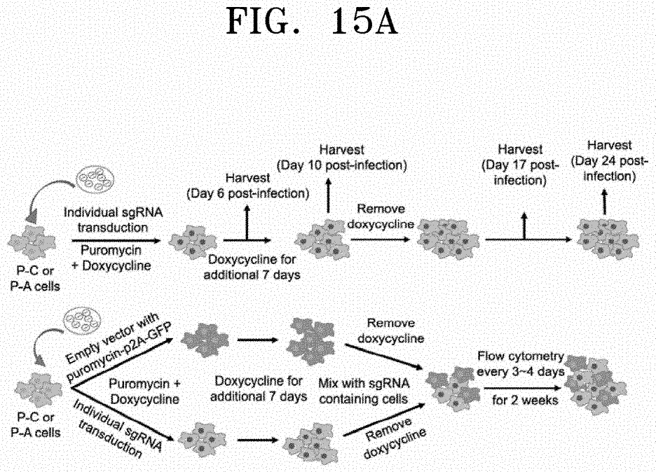

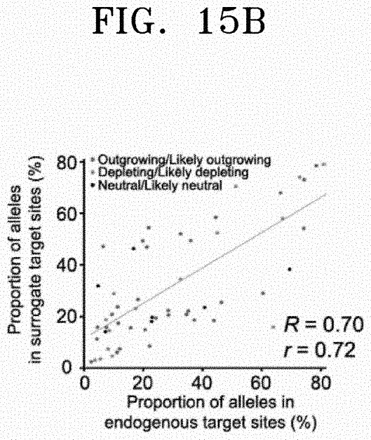

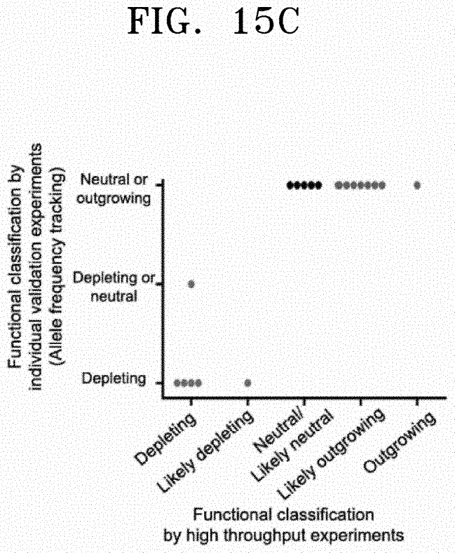

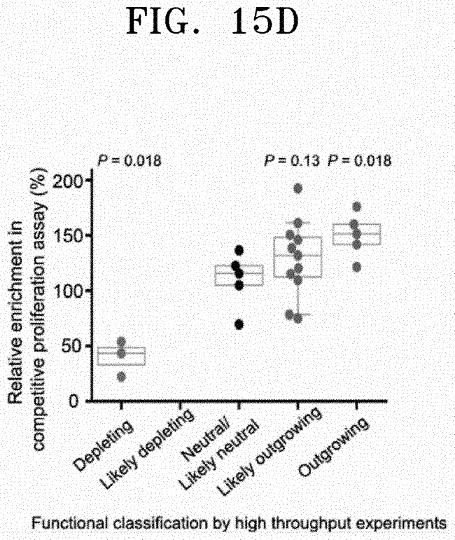

To compare the LFCs of the sgRNAs according to the base editing efficiency (FIG. 9) and the enrichment values of the target sgRNA and non-targeting sgRNA control (FIG. 15D), the two-tailed Student's t-test was used. Statistical significance was calculated using PASW Statistics (version 18.0, IBM). One-way analysis of variance followed by Dunn's post hoc test was used to compare base conversion efficiency among score bins. To determine the fraction of sgRNAs predicted to introduce mutations in common essential genes (CEGs) or cancer gene census (CGC) genes among all classified sgRNAs, the Fisher's exact test was performed using the scipy.stats.fisher_exact function of the Python library.

### 18. Data Availability and Code Availability

The deep sequencing data from this study was submitted to the NCBI Sequence Read Archive under accession number PRJNA667758. The custom Python scripts used for the generation of the MAGECK input file using UMIs are available on github (https://github.com/oreolic/CancerLibrary).

### Embodiments

### Embodiment 1. Generating Cancer-Associated Mutations Using Base Editors

To introduce cancer-associated transition mutations into endogenous target sequences using CBE and ABE, cell lines were generated first that express CBE or ABE in a doxycycline-responsive manner. HBEC3OKT cells are immortalized non-tumorigenic bronchial epithelial cells derived from normal lung cells. HBEC3OKT cells were used as precancerous cells that lentivirally express a short hairpin RNA (shRNA) targeting TP53 (HBEC3OKT-shTP53; hereafter, for brevity, P cells). Although P cells express only low levels of TP53 mRNA, gene set enrichment analysis showed that the p53 pathway was upregulated. Similar to HBEC3OKT cells, P cells require epidermal growth factor (EGF) for cell expansion and are non-tumorigenic, and lentiviral vectors expressing reverse tetracycline-controlled transactivator (rtTA) and a base editor (CBE or ABE) shown in FIG. 1 were sequentially transduced into P cells. The resulting cell lines, which express CBE or ABE in a doxycycline-inducible manner, were named P-C or P-A cells, respectively.

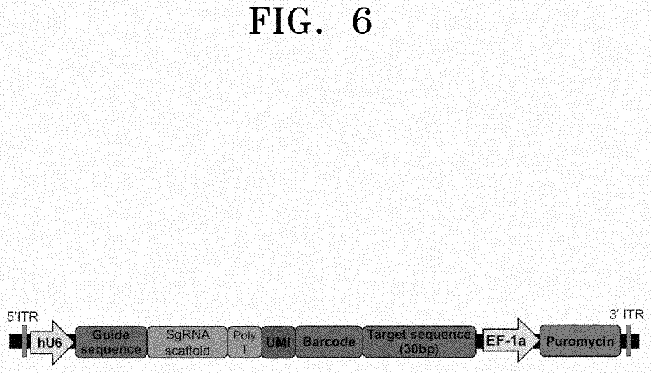

To identify target sequences that can be modified by CBE or ABE to contain transition mutations observed in human cancer tissues, 84,806 C>T and G>A single-nucleotide variants (SNVs) and 23,176 A>G and T>C SNVs that can be respectively generated by CBE and ABE at high predicted efficiencies using 80,203 and 23,008 sgRNAs were identified by using the Catalogue of Somatic Mutations in Cancer (COSMIC). In addition, two negative control groups of sgRNAs were added: the first group contained sgRNAs that do not target any sequences in the human genome (hereafter, non-targeting sgRNAs or NT), and the second group consisted of sgRNAs that, with CBE or ABE, would induce synonymous mutations. As a result of this process shown in FIG. 2, 83,731 and 23,613 sgRNAs for CBE and ABE, respectively, were identified. To monitor base editing efficiencies and outcomes, an sgRNA-encoding lentiviral vector corresponding to a surrogate target sequence was used, and the vector used in the experiment is schematically shown in FIG. 6.

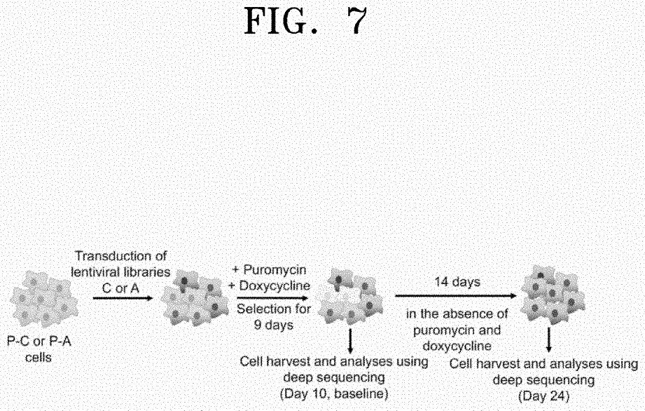

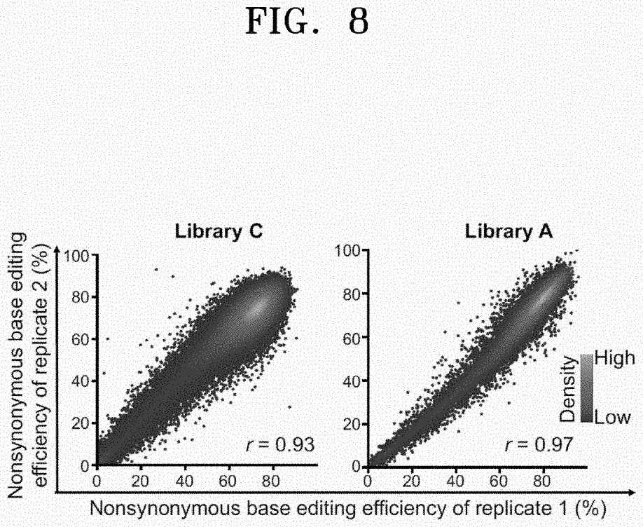

Lentiviral libraries, respectively named libraries C and A, of the 83,731 (for CBE) and 23,613 (for ABE) pairs of an sgRNA-encoding sequence and a target sequence were generated. The frequency of shuffling of barcodes and sgRNA-encoding sequences was about 4.3%, which did not substantially affect the functional evaluations. In addition, an 8-nucleotide (nt) long unique molecular identifier (UMI) was added between the sgRNA-encoding and target sequences in both libraries for tracking of transduced cells and subsequent analyses. The libraries C and A were respectively transduced into P-C and P-A cells in duplicate. The culture medium including the cells were supplemented with doxycycline to induce CBE or ABE expression. This series of processes is shown in FIG. 7. When the base editing efficiencies at the integrated target sequences were measured at day 10 after the initial transduction, the efficiencies were found to be high (FIGS. 4A and 4B); the median efficiencies at positions 4, 5, 6, and 7 were 37%, 59%, 61%, and 53% for CBE and 16%, 68%, 68%, and 59% for ABE. Amino acid-changing or nonsynonymous editing efficiencies in independent biological replicates were compared and the results are shown in FIG. 8, and as identified in FIG. 8, high correlations with Pearson correlation coefficients of 0.93 and 0.97 were observed. Very low levels of indels were observed at the integrated target sequences. Thus, we did not use these sgRNAs as negative controls in subsequent analysis.

Next, the relationship between the base editing efficiency at integrated target sequences and phenotypic changes were investigated. Using 190 unique sgRNAs targeting 65 curated essential genes in the C2 library generated as described above, robust depletion of sgRNA-transduced cells was exhibited as shown in FIG. 9, when the nonsynonymous base editing efficiency in the surrogate sequences was over 60%. Therefore, sgRNAs associated with a less than 60% base editing efficiency in surrogate sequences could result in insufficient base editing at the endogenous target sites, which could mask a possible outgrowing or depleting phenotype associated with such sgRNAs. When the relationship between the base editing efficiencies at endogenous sites, as a parameter for the growth phenotype, such as an increase or decrease in proliferation and survival, and the log-fold changes (LFCs) of the corresponding sgRNAs were mathematically calculated, the LFC and base editing efficiency were correlated. In addition, when base editing efficiencies were lower than 60%, a larger percentage of sgRNAs inducing stop codons in essential genes were classified as neutral than when the efficiencies were higher than 60%. Thus, those inefficient sgRNAs with an insufficient number of UMIs at day 10 were filtered out from functional classifications.

### Embodiment 2. Functional Classification of Cancer-Associated Mutations

To evaluate the functional effects of the variants generated by CBE and ABE on cell proliferation and survival, these mutation-containing cell populations were cultured in the absence of doxycycline for 14 days. Genomic DNA was isolated from the cell populations at day 10 (baseline) and day 24 after the initial transduction of libraries C and A, and subjected to deep sequencing to evaluate the relative frequencies of sgRNA and target sequence pairs and UMIs. Median LFCs and P-values for each sgRNA were calculated. Based on the −log₁₀(P-value) and the median LFC of each sgRNA, the sgRNAs were functionally classified into depleting, likely depleting, likely neutral (possibly depleting), neutral, likely neutral (possibly outgrowing), likely outgrowing, and outgrowing using the distribution of control non-targeting sgRNAs (FIGS. 5 and 10).

In addition to changes in the abundance of each UMI (i.e., the LFC in the RPM (reads per million) of a UMI), the number of UMIs for each sgRNA (i.e., the LFC in the UMI counts per million (CPM)) was also utilized for functional classification. The number of UMIs for each sgRNA decreases when an sgRNA-induced mutation is depleting. Thus, the number of UMIs for each sgRNA was used as an additional parameter to increase the accuracy of hit calling in Cas9-based screening. To reduce the number of false depleting or outgrowing sgRNAs in the classifications, sgRNAs that meet the criteria for depleting or outgrowing with respect to the LFC in the RPM and P-value as likely depleting or likely outgrowing when the LFC in the UMI CPM did not suggest depletion or outgrowth, respectively.

### Embodiment 3. Confirmation of Functional Evaluation Method at Different Scales

Experiments were performed to confirm whether the evaluation method of a large amount of mutation data confirmed through the above embodiment is applicable at various scales and whether the classification result is reproducible using an independent library, and the procedure of this experiment is shown in FIGS. 3A and 3B. For this purpose, three smaller libraries (containing 3,261 and 3,170 unique sgRNAs for CBE and 1,595 unique sgRNAs for ABE) named C1, C2 and A1, respectively, were prepared (FIGS. 3A and 3B). As found in FIG. 3C, a high correlation was identified between nonsynonymous base editing efficiency in the same integrated target sequences of libraries C, C1 and C2 and libraries A and A1. In addition, such a relationship was also confirmed in FIG. 11.

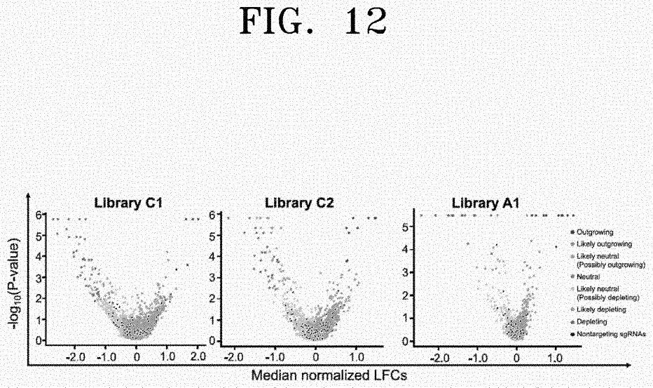

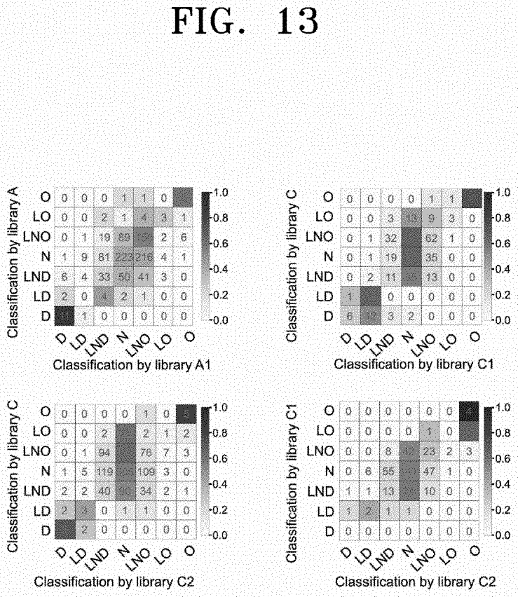

Therefore, functional classification of the sgRNAs of libraries C1, C2 and A1 was performed using the method confirmed in Embodiment 2 and the experimental examples, and this is shown in FIG. 12. As found in FIG. 12 , functional classification of variants of large libraries (C and A) was confirmed to be well compatible even in small libraries. Accordingly, as found in FIG. 13, it was finally identified that the functional evaluation method may be effectively utilized even when the size of the library of the functional evaluation is reduced.

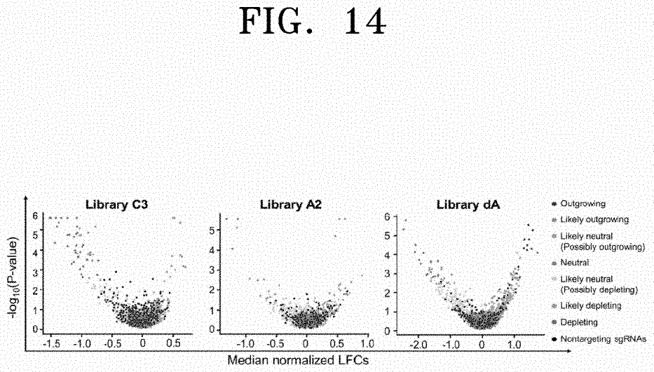

### Embodiment 4. Confirmation of Function Evaluation Method Using Additional Libraries

Whereas most nonsense mutations lead to loss-of-function, the functional effects of missense mutations are more difficult to predict. Thus, two more ABE libraries were additionally generated to confirm the functional evaluation method of mutations. One is dA (drivers for ABE) library that can induce 2,797 missense transition mutations observed in 262 driver genes, and another is A2 library that can induce 1,468 missense transition mutations observed in 627 tumor suppressor genes. In addition, another library named C3 was generated to induce 1,080 missense transition mutations and 83 nonsense mutations observed in 116 tumor suppressor genes, and the method was confirmed by the experimental method as a method for functional evaluation of variants, and the results of these experiments are shown in FIG.14. As found in FIG. 14, the method was confirmed to be useful for functional evaluations of variants through the experiments using three additional libraries.

### Embodiment 5. Final Confirmation of Evaluation Method for Functional Classification of Variants Based on Integrated Results

The results from libraries C, C1, C2, C3, A, A1, A2, and dA were integrated and an experiment was performed to identify whether the classifications of the sgRNAs and associated protein variants may be made clear.

A total of 68,070 sgRNAs were classified as follows: 282 depleting, 691 likely depleting, 14,689 likely neutral (possibly depleting), 34,714 neutral, 17,248 likely neutral (possibly outgrowing), 409 likely outgrowing, and 37 outgrowing.

Analysis of the surrogate target sequences allowed identification of which sgRNAs mainly induced a single protein variant in a highly efficient manner. In these cases, the phenotype induced by the sgRNA may be attributed to that major protein variant, which is called a “primary” protein variant for the sgRNA in the disclosure. A protein variant was classified as a “primary” protein variant when its relative frequency among all the base editor-generated protein variants was higher than 75% (the frequency of the primary variant was at least three times higher than the combined frequencies of the remaining base-edited variants).

Thus, functional classifications for 29,060 protein variants generated by transition mutations were provided (FIG. 5): 123 depleting, 304 likely depleting, 6,281 likely neutral (possibly depleting), 14,949 neutral, 7,228 likely neutral (possibly outgrowing), 157 likely outgrowing, and 18 outgrowing.

Each of the remaining 39,012 functionally classified sgRNAs induced either a single primary variant with two amino acid changes (12,820 sgRNAs) or a group of variants without a primary variant (26,192 sgRNAs). In the variants induced by the 39,012 sgRNAs, the frequency of each variant in the variant group and the functional effects of the variant group, which are determined by analysis of the functional effects of single amino acid changes, were identified. The surrogate target sequences could be, albeit at low accuracy, informative and was confirmed to assist in predicting the functional effects of single amino acid changes, especially when the phenotypes induced by the sgRNAs are classified as neutral. Most sgRNAs (77%, 20,138) out of the 26,192 sgRNAs that induced multiple variants without a primary variant were confirmed to generate two protein variants when variants whose frequencies are higher than 10%. were used to evaluate. Thus, phenotypes of these 20,138 sgRNAs are confirmed to be attributable to either or both of the two corresponding protein variants.

### Embodiment 6. Validation of Functional High-Throughput Data Classification Evaluation Method Through Functional Identification of Individual Mutations

To validate the evaluation method based on the results of the high-throughput experiments, experiments were conducted to identify whether actual predictions are possible by independently identifying individual effects of the variants generated by base editing. 28 sgRNAs used for the high-throughput evaluations were selected, and these sgRNAs were individually delivered into P-C or P-A cells via lentiviral transduction. The transduced cells were cultured with doxycycline for 7 days to induce base editing and incubated for an additional 14 days in the absence of doxycycline. The cells were harvested and analyzed at 6, 10, 17, and 24 days post-infection to track individual allele frequencies after delivering sgRNAs, a series of experimental procedures are shown in FIG. 15A, and the analysis results thereof are shown in FIG. 15B.

As confirmed in FIG. 15B, high correlations were observed between the frequencies of 61 base-edited alleles induced by the 20 selected sgRNAs at integrated target sequences of the high-throughput experiments according to an embodiment and the frequencies at the endogenous target sites in the independent individual experiments. Pearson r=0.72, Spearman R=0.70).

In addition, an individual function of an sgRNA was classified by observing that the frequencies of variants generated by the base editing increased, remained unchanged, or decreased through deep sequencing analysis. Specifically, an sgRNA was classified as depleting when the base-edited variant frequency decreased and the wild-type sequence frequency increased over time after day 10. When the base-edited variant frequency increased and the wild-type sequence frequency decreased, the relevant sgRNA was classified as outgrowing or neutral. When the frequencies of base-edited variants and wild-type sequences were unchanged over time after day 10, the sgRNAs were classified as neutral or depleting. The results of this functional classification of individual sgRNAs based on the frequencies of variant and wild-type sequences and the results from the high-throughput evaluations according to the embodiment were analyzed, and as a result, as confirmed in FIG. 15C, the results of the high-throughput classification evaluation method according to the method of the embodiment was found to be consistent with the results of the individual evaluation methods (FIG. 4C and Extended Data FIG. 8). However, because it is difficult to distinguish outgrowing and neutral phenotypes using such variant frequency tracking, competitive proliferation assays were next performed, and the experiment of the method at the bottom of FIG. 15A was performed to compare the proliferation of sgRNA-transduced and non-transduced cells. sgRNAs were classified based on the enrichment or depletion of the sgRNA-transduced cells over time as compared to those transduced with non-targeting sgRNAs, and the experimental results thereof are shown in FIG. 15D. As found in FIG. 15D, flow cytometry showed that the classifications based on this assay were compatible with the classifications of the high-throughput evaluations of the embodiment, and finally, it was confirmed that the high-throughput evaluation method according to the embodiment may evaluate large amounts of data and perform functional classification according to sgRNA with high accuracy and give results compatible to those of the individual classification method.

### Embodiment 7. Identification of Function of Anticancer Drug Resistance-Related Mutations According to EGF Signaling

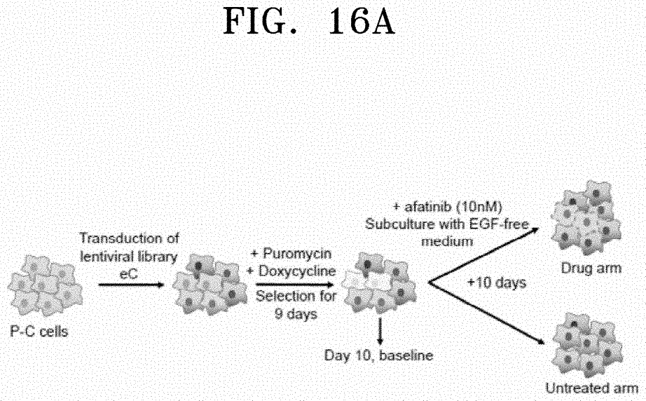

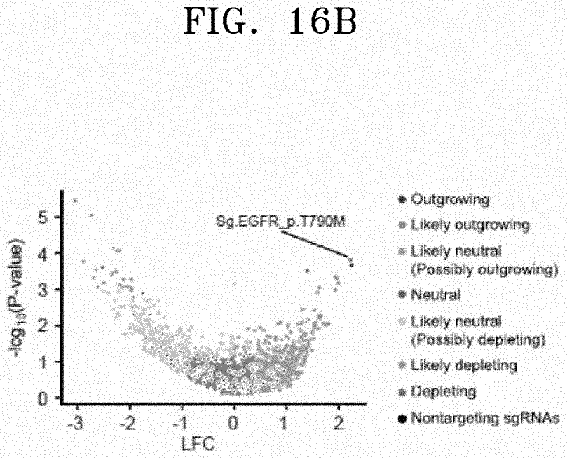

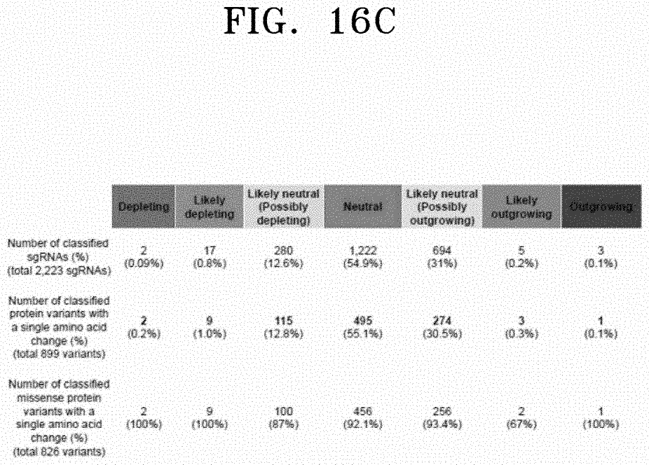

The evaluation method according to the embodiment is based on evaluations of cell proliferation and viability. Given that one of the most important hallmarks of cancer is self-sufficiency in growth signals, the cells' dependency on a growth signal, EGF, which is required for the proliferation of P cells, were evaluated. A library named eC (epidermal growth factor-CBE) was generated to induce 3,967 transition mutations observed in 162 genes related to the EGF/EGF receptor (EGFR) signaling pathway. The eC library was transduced into P-C cells and base editing was induced by the addition of doxycycline. The cell population was split into an EGF depletion arm, in which EGF was removed and 10 nM of the EGFR inhibitor afatinib was added, and an untreated control arm, after which both arms were cultured for an additional ten days, and the series of experimental procedures are shown in FIG. 16A. Similar to experiments conducted in the above embodiment, sgRNAs were functionally classified by comparing the number of cells in the EGF depletion group with the number of cells in the control group, and the observed results are shown in FIG. 16B and summarized in FIG. 16C.

As found in FIGS. 16B and 16C, as a result of evaluating the gene editing outcomes at the integrated target sequences according to the method of the embodiment, functional classifications of a total of 899 protein variants with a single amino acid change were found to be possible. Among the variants classified above, one variant that conferred resistance to the anticancer drug afatinib, EGFR p.T790M, was found, and these mutations were accurately identified to be growth mutations that actively proliferate despite drug administration. In addition, two depleting variants SH3GL3_p.D169N and PIK3C2B_p.E650K, and 495 neutral variants were identified. The high-throughput evaluation identified in the embodiment revealed that EGFR_p.P753S, which is known as VUS, is associated with a likely depleting phenotype. And the finding was compatible with a case report that a patient with this variant showed a dramatically positive response to therapy with cetuximab, an EGFR inhibitor, for treating cutaneous squamous cell carcinoma.

### Embodiment 8. Confirmation of Function of Variants Identified Through High-Throughput Functional Evaluation Method for Variants

Among the protein variants classified by the functional evaluation method confirmed in the embodiment, the majority of them (28,458/29,060=98%) were confirmed to be classified as neutral (14,949, 51%) or likely neutral (13,509, 46%).

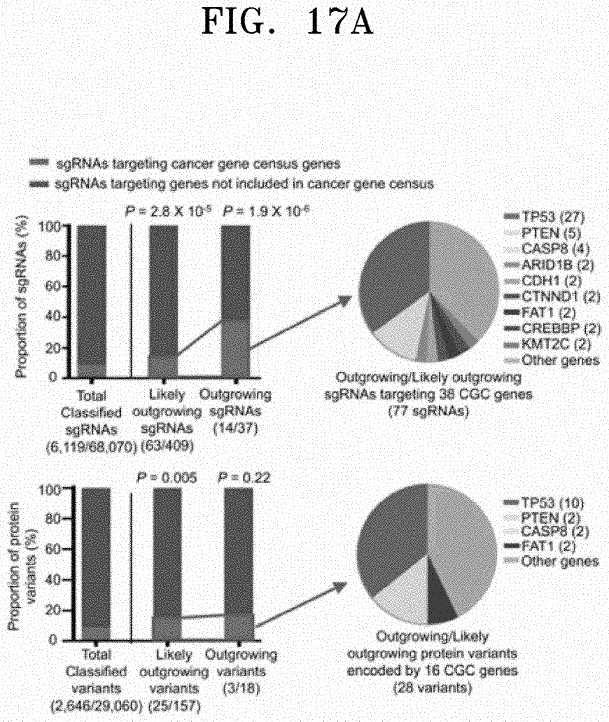

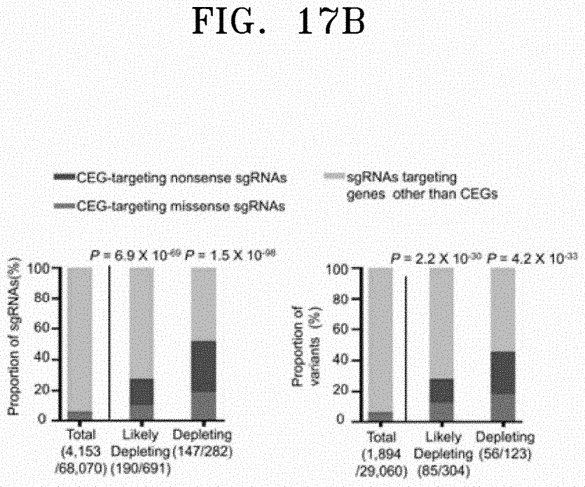

Notable gene groups related to the outgrowing phenotype included those in the Cancer Gene Census (CGC), and their functional classification is shown in FIG. 17A (top). As found in FIG. 17A, among the 68,070 functionally classified sgRNAs, 9.0% (6,119) targeted CGC genes. However, outgrowing in the CGC gene fraction was found to be 15% (63/409) (P-value=2.8×10⁻⁵), and likely outgrowing was found to be 38% (14/37) (P-value=1.9×10⁻⁶). Among the sgRNAs targeting CGC genes in these two groups, the largest fraction, 35% (27/77), was found to target TP53.

The same analysis was performed using 29,060 functionally classified protein variants, and the results are shown at the bottom of FIG. 17A. As found in FIG. 17A, the gene most related to variants classified as outgrowing and likely outgrowing among CGC genes was found to be TP53 (36% (10/28)).

Notable gene groups related to the depleting phenotype were identified, and the results of the confirmation are shown in the left side of FIG. 17B. As found in FIG. 17B, notable gene groups related to the depleting phenotype included common essential genes, among the sgRNAs, 52% (147/282) (P value=6.9×10⁻⁶⁹) of the depleting sgRNAs and 27% (190/691) (P value=1.5×10⁻⁹⁸) of the likely depleting sgRNAs were found to be associated with common essential genes, whereas only 6.1% (4,153/68,070) of all functionally classified sgRNAs were found to target common essential genes.

Moreover, an analysis was performed using 29,060 functionally classified protein variants instead of sgRNAs, and the results are shown on the right side of FIG. 17B. As found in FIG. 17B, results similar to the those found when conducting experiments with sgRNA were identified.

As results of the experiments identified in individual mutations, the p.Y727C mutation affecting EGFR was found to exhibit an outgrowing phenotype both in the high-throughput mutation analyses according to an embodiment and individual validation experiments. Given that EGFR activation induces cell proliferation and survival, the p.Y727C mutation, which was found to be VUS by ClinVar, was found to be classified as a gain-of-function mutation according to the method of the embodiment.

Although the role of PHLDA1 mutations in cancer has not been well-evaluated, but both the high-throughput mutation analyses according to the embodiment and individual validation experiments suggested that the p.Q201* and p.Y249C variants cause an outgrowing and a likely outgrowing phenotype, respectively, these mutations were found to increase cell survival and proliferation. Furthermore, IRF6 is known to have tumor suppressor activity in squamous cell carcinomas, and the p.Y97C mutation in IRF6 was known to be the cause of Van der Woude syndrome, characterized by a cleft lip and palate. Therefore, high-throughput mutation analysis and individual validation experiments were performed, and p.Y97C was confirmed to exhibit outgrowing phenotypes in both experimental results. CASP8 is related to apoptosis and has been known to be a tumor suppressor, and the high-throughput mutation analysis according to an embodiment showed that two variants, whose functions are unknown, p.S158F and p.Y507C, cause outgrowing and likely outgrowing phenotypes, respectively. An individual validation experiment confirmed the outgrowing phenotype of p.S158F. CREBBP has been known to be a tumor suppressor against small cell lung carcinoma, leukemia, and lymphoma and the p.Y1482H variant has been known to be a loss-of-function mutation in lymphoma. In line with these findings, the high-throughput mutant evaluation according to an embodiment revealed that p.Y1482H causes an outgrowing phenotype.

What is claimed is:

1. A method of evaluating functions of cancer mutations comprising:
generating a cell library including a nucleotide sequence encoding a guide RNA, a unique molecular identifier (UMI) nucleotide sequence, and an oligonucleotide including a target nucleotide sequence targeted by the guide RNA;
transducing the cell library into cells expressing the base editors and culturing the cells;
harvesting the transduced cells after culturing, performing deep sequencing, and measuring the data of the base editing efficiency and the frequency level of protein mutations due to base editing; and
analyzing the measured data to evaluate the function of the mutation introduced into the cell library.

2. The method of evaluating functions of cancer mutations of claim 1, wherein the base editors are cytosine base editors (CBEs) and adenine base editors (ABEs).

3. The method of evaluating functions of cancer mutations of claim 1, wherein the analysis of the measured data is classifying data as valid when the efficiency of base conversion and the frequency of protein mutations due to the base conversion meet criteria, and analyzing the same.

4. The method of evaluating functions of cancer mutations of claim 3, wherein the criteria are
1) The efficiency of base conversion in the target sequence is 60% or more; and
2) the frequency of the intended protein mutation is 75% or more compared to the frequency of the unintended protein mutation.

5. The method of evaluating functions of cancer mutations of claim 1, wherein the evaluating the function of the mutation includes classifying each mutation as an outgrowing or depletion (depleting) mutation.

6. An evaluation system for cancer mutations comprising:
an information input unit for receiving data of the base conversion efficiency by base editors and the frequency level of protein mutation caused by a base conversion;
a data classification unit for classifying data as valid data in case the data received from the information input unit meet criteria; and
a data evaluation unit that analyzes the data classified by the data classification unit and analyzes the measured data to evaluate the function of the mutation.

7. The evaluation system for cancer mutations of claim 6, wherein the base editors are cytosine base editors (CBEs) and adenine base editors (ABEs).

8. The evaluation system for cancer mutations of claim 6, wherein the criteria are
1) The efficiency of base conversion in the target sequence is 60% or more; and
2) the frequency of the intended protein mutation is 75% or more compared to the frequency of the unintended protein mutation.

9. The evaluation system for cancer mutations of claim 6, wherein the data of the base conversion efficiency by base editors and the frequency level of protein mutation through the base conversion are obtained by:
generating a cell library including a nucleotide sequence encoding a guide RNA, a unique molecular identifier (UMI) nucleotide sequence, and an oligonucleotide including a target nucleotide sequence targeted by the guide RNA;
transducing the cell library into cells expressing the base editors and culturing the cells; and
harvesting the transduced cells after culturing, performing deep sequencing, and measuring the data of the base editing efficiency and the frequency level of protein mutations due to base editing.

10. A computer-readable recording medium in which is recorded a program for executing the method according to claim 1 by a computer.
